SEQUENCE LISTING (162 sequences)

SEQ ID NO: 1 - DNA, 10 bp, Artificial sequence
  derived from a Kozak sequence
  misc_feature (8)..(8): n = A or U
ssmrccangg

SEQ ID NO: 2 - DNA, 10 bp, Artificial sequence
  derived from a Kozak sequence
ssmrccatgg

SEQ ID NO: 3 - DNA, 10 bp, Artificial sequence
  derived from a Kozak sequence
ssmrccaugg

SEQ ID NO: 4 - DNA, 10 bp, Artificial sequence
  derived from a Kozak sequence
ggcgccatgg

SEQ ID NO: 5 - DNA, 10 bp, Artificial sequence
  derived from a Kozak sequence
ggcaccatgg

SEQ ID NO: 6 - DNA, 10 bp, Artificial sequence
  derived from a Kozak sequence
ggagccatgg

SEQ ID NO: 7 - DNA, 10 bp, Artificial sequence
  derived from a Kozak sequence
ggaaccatgg

SEQ ID NO: 8 - DNA, 10 bp, Artificial sequence
  derived from a Kozak sequence
gccgccatgg

SEQ ID NO: 9 - DNA, 10 bp, Artificial sequence
  derived from a Kozak sequence
gccaccatgg

SEQ ID NO: 10 - DNA, 10 bp, Artificial sequence
  derived from a Kozak sequence
gcagccatgg

SEQ ID NO: 11 - DNA, 10 bp, Artificial sequence
  derived from a Kozak sequence
gcaaccatgg

SEQ ID NO: 12 - DNA, 10 bp, Artificial sequence
  derived from a Kozak sequence
cgcgccatgg

SEQ ID NO: 13 - DNA, 10 bp, Artificial sequence
  derived from a Kozak sequence
cgcaccatgg

SEQ ID NO: 14 - DNA, 10 bp, Artificial sequence
  derived from a Kozak sequence
cgagccatgg

SEQ ID NO: 15 - DNA, 10 bp, Artificial sequence
  derived from a Kozak sequence
cgaaccatgg

SEQ ID NO: 16 - DNA, 10 bp, Artificial sequence
  derived from a Kozak sequence
cccgccatgg

SEQ ID NO: 17 - DNA, 10 bp, Artificial sequence
  derived from a Kozak sequence
cccaccatgg

SEQ ID NO: 18 - DNA, 10 bp, Artificial sequence
  derived from a Kozak sequence
ccagccatgg

SEQ ID NO: 19 - DNA, 10 bp, Artificial sequence
  derived from a Kozak sequence
ccaaccatgg

SEQ ID NO: 20 - RNA, 10 nt, Artificial sequence
  derived from a Kozak sequence
ggcgccaugg

SEQ ID NO: 21 - RNA, 10 nt, Artificial sequence
  derived from a Kozak sequence
ggcaccaugg

SEQ ID NO: 22 - RNA, 10 nt, Artificial sequence
  derived from a Kozak sequence
ggagccaugg

SEQ ID NO: 23 - RNA, 10 nt, Artificial sequence
  derived from a Kozak sequence
ggaaccaugg

SEQ ID NO: 24 - RNA, 10 nt, Artificial sequence
  derived from a Kozak sequence
gccgccaugg

SEQ ID NO: 25 - RNA, 10 nt, Artificial sequence
  derived from a Kozak sequence
gccaccaugg

SEQ ID NO: 26 - RNA, 10 nt, Artificial sequence
  derived from a Kozak sequence
gcagccaugg

SEQ ID NO: 27 - RNA, 10 nt, Artificial sequence
  derived from a Kozak sequence
gcaaccaugg

SEQ ID NO: 28 - RNA, 10 nt, Artificial sequence
  derived from a Kozak sequence
cgcgccaugg

SEQ ID NO: 29 - RNA, 10 nt, Artificial sequence
  derived from a Kozak sequence
cgcaccaugg

SEQ ID NO: 30 - RNA, 10 nt, Artificial sequence
  derived from a Kozak sequence
cgagccaugg

SEQ ID NO: 31 - RNA, 10 nt, Artificial sequence
  derived from a Kozak sequence
cgaaccaugg

SEQ ID NO: 32 - RNA, 10 nt, Artificial sequence
  derived from a Kozak sequence
cccgccaugg

SEQ ID NO: 33 - RNA, 10 nt, Artificial sequence
  derived from a Kozak sequence
cccaccaugg

SEQ ID NO: 34 - RNA, 10 nt, Artificial sequence
  derived from a Kozak sequence
ccagccaugg

SEQ ID NO: 35 - RNA, 10 nt, Artificial sequence
  derived from a Kozak sequence
ccaaccaugg

SEQ ID NO: 36 - DNA, 10 bp, Artificial sequence
  derived from a Kozak sequence
  misc_feature (8)..(8): n = A or U
ssmrccangt

SEQ ID NO: 37 - DNA, 12 bp, Artificial sequence
  derived from a Kozak sequence
  misc_feature (8)..(8): n = A or U
  misc_feature (10)..(10): n = A or U
ssmrccanangg

SEQ ID NO: 38 - DNA, 10 bp, Artificial sequence
  derived from a Kozak sequence
ggcgccatgt

SEQ ID NO: 39 - DNA, 10 bp, Artificial sequence
  derived from a Kozak sequence
ggcaccatgt

SEQ ID NO: 40 - DNA, 10 bp, Artificial sequence
  derived from a Kozak sequence
ggagccatgt

SEQ ID NO: 41 - DNA, 10 bp, Artificial sequence
  derived from a Kozak sequence
ggaaccatgt

SEQ ID NO: 42 - DNA, 10 bp, Artificial sequence
  derived from a Kozak sequence
gccgccatgt

SEQ ID NO: 43 - DNA, 10 bp, Artificial sequence
  derived from a Kozak sequence
gccaccatgt

SEQ ID NO: 44 - DNA, 10 bp, Artificial sequence
  derived from a Kozak sequence
gcagccatgt

SEQ ID NO: 45 - DNA, 10 bp, Artificial sequence
  derived from a Kozak sequence
gcaaccatgt

SEQ ID NO: 46 - DNA, 10 bp, Artificial sequence
  derived from a Kozak sequence
cgcgccatgt

SEQ ID NO: 47 - DNA, 10 bp, Artificial sequence
  derived from a Kozak sequence
cgcaccatgt

SEQ ID NO: 48 - DNA, 10 bp, Artificial sequence
  derived from a Kozak sequence
cgagccatgt

SEQ ID NO: 49 - DNA, 10 bp, Artificial sequence
  derived from a Kozak sequence
cgaaccatgt

SEQ ID NO: 50 - DNA, 10 bp, Artificial sequence
  derived from a Kozak sequence
cccgccatgt

SEQ ID NO: 51 - DNA, 10 bp, Artificial sequence
  derived from a Kozak sequence
cccaccatgt

SEQ ID NO: 52 - DNA, 10 bp, Artificial sequence
  derived from a Kozak sequence
ccagccatgt

SEQ ID NO: 53 - DNA, 10 bp, Artificial sequence
  derived from a Kozak sequence
ccaaccatgt

SEQ ID NO: 54 - DNA, 12 bp, Artificial sequence
  derived from a Kozak sequence
ggcgccatatgg

SEQ ID NO: 55 - DNA, 12 bp, Artificial sequence
  derived from a Kozak sequence
ggcaccatatgg

SEQ ID NO: 56 - DNA, 12 bp, Artificial sequence
  derived from a Kozak sequence
ggagccatatgg

SEQ ID NO: 57 - DNA, 12 bp, Artificial sequence
  derived from a Kozak sequence
ggaaccatatgg

SEQ ID NO: 58 - DNA, 12 bp, Artificial sequence
  derived from a Kozak sequence
gccgccatatgg

SEQ ID NO: 59 - DNA, 12 bp, Artificial sequence
  derived from a Kozak sequence
gccaccatatgg

SEQ ID NO: 60 - DNA, 12 bp, Artificial sequence
  derived from a Kozak sequence
gcagccatatgg

SEQ ID NO: 61 - DNA, 12 bp, Artificial sequence
  derived from a Kozak sequence
gcaaccatatgg

SEQ ID NO: 62 - DNA, 12 bp, Artificial sequence
  derived from a Kozak sequence
cgcgccatatgg

SEQ ID NO: 63 - DNA, 12 bp, Artificial sequence
  derived from a Kozak sequence
cgcaccatatgg

SEQ ID NO: 64 - DNA, 12 bp, Artificial sequence
  derived from a Kozak sequence
cgagccatatgg

SEQ ID NO: 65 - DNA, 12 bp, Artificial sequence
  derived from a Kozak sequence
cgaaccatatgg

SEQ ID NO: 66 - DNA, 12 bp, Artificial sequence
  derived from a Kozak sequence
cccgccatatgg

SEQ ID NO: 67 - DNA, 12 bp, Artificial sequence
  derived from a Kozak sequence
cccaccatatgg

SEQ ID NO: 68 - DNA, 12 bp, Artificial sequence
  derived from a Kozak sequence
ccagccatatgg

SEQ ID NO: 69 - DNA, 12 bp, Artificial sequence
  derived from a Kozak sequence
ccaaccatatgg

SEQ ID NO: 70 - RNA, 10 nt, Artificial sequence
  derived from a Kozak sequence
ggcgccaugu

SEQ ID NO: 71 - RNA, 10 nt, Artificial sequence
  derived from a Kozak sequence
ggcaccaugu

SEQ ID NO: 72 - RNA, 10 nt, Artificial sequence
  derived from a Kozak sequence
ggagccaugu

SEQ ID NO: 73 - RNA, 10 nt, Artificial sequence
  derived from a Kozak sequence
ggaaccaugu

SEQ ID NO: 74 - RNA, 10 nt, Artificial sequence
  derived from a Kozak sequence
gccgccaugu

SEQ ID NO: 75 - RNA, 10 nt, Artificial sequence
  derived from a Kozak sequence
gccaccaugu

SEQ ID NO: 76 - RNA, 10 nt, Artificial sequence
  derived from a Kozak sequence
gcagccaugu

SEQ ID NO: 77 - RNA, 10 nt, Artificial sequence
  derived from a Kozak sequence
gcaaccaugu

SEQ ID NO: 78 - RNA, 10 nt, Artificial sequence
  derived from a Kozak sequence
cgcgccaugu

SEQ ID NO: 79 - RNA, 10 nt, Artificial sequence
  derived from a Kozak sequence
cgcaccaugu

SEQ ID NO: 80 - RNA, 10 nt, Artificial sequence
  derived from a Kozak sequence
cgagccaugu

SEQ ID NO: 81 - RNA, 10 nt, Artificial sequence
  derived from a Kozak sequence
cgaaccaugu

SEQ ID NO: 82 - RNA, 10 nt, Artificial sequence
  derived from a Kozak sequence
cccgccaugu

SEQ ID NO: 83 - RNA, 10 nt, Artificial sequence
  derived from a Kozak sequence
cccaccaugu

SEQ ID NO: 84 - RNA, 10 nt, Artificial sequence
  derived from a Kozak sequence
ccagccaugu

SEQ ID NO: 85 - RNA, 10 nt, Artificial sequence
  derived from a Kozak sequence
ccaaccaugu

SEQ ID NO: 86 - RNA, 12 nt, Artificial sequence
  derived from a Kozak sequence
ggcgccauaugg

SEQ ID NO: 87 - RNA, 12 nt, Artificial sequence
  derived from a Kozak sequence
ggcaccauaugg

SEQ ID NO: 88 - RNA, 12 nt, Artificial sequence
  derived from a Kozak sequence
ggagccauaugg

SEQ ID NO: 89 - RNA, 12 nt, Artificial sequence
  derived from a Kozak sequence
ggaaccauaugg

SEQ ID NO: 90 - RNA, 12 nt, Artificial sequence
  derived from a Kozak sequence
gccgccauaugg

SEQ ID NO: 91 - RNA, 12 nt, Artificial sequence
  derived from a Kozak sequence
gccaccauaugg

SEQ ID NO: 92 - RNA, 12 nt, Artificial sequence
  derived from a Kozak sequence
gcagccauaugg

SEQ ID NO: 93 - RNA, 12 nt, Artificial sequence
  derived from a Kozak sequence
gcaaccauaugg

SEQ ID NO: 94 - RNA, 12 nt, Artificial sequence
  derived from a Kozak sequence
cgcgccauaugg

SEQ ID NO: 95 - RNA, 12 nt, Artificial sequence
  derived from a Kozak sequence
cgcaccauaugg

SEQ ID NO: 96 - RNA, 12 nt, Artificial sequence
  derived from a Kozak sequence
cgagccauaugg

SEQ ID NO: 97 - RNA, 12 nt, Artificial sequence
  derived from a Kozak sequence
cgaaccauaugg

SEQ ID NO: 98 - RNA, 12 nt, Artificial sequence
  derived from a Kozak sequence
cccgccauaugg

SEQ ID NO: 99 - RNA, 12 nt, Artificial sequence
  derived from a Kozak sequence
cccaccauaugg

SEQ ID NO: 100 - RNA, 12 nt, Artificial sequence
  derived from a Kozak sequence
ccagccauaugg

SEQ ID NO: 101 - RNA, 12 nt, Artificial sequence
  derived from a Kozak sequence
ccaaccauaugg

SEQ ID NO: 102 - DNA, 714 bp, Artificial sequence
  eGFP protein
gtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 103 - DNA, 888 bp, Mus musculus
atggaacaccagctcctgtgctgcgaagtggagaccatccgccgcgcgtaccctgacaccaatctcctcaacgaccgggtgctgcgagccatgctcaagacggaggagacctgtgcgccctccgtatcttacttcaagtgcgtgcagaaggagattgtgccatccatgcggaaaatcgtggccacctggatgctggaggtctgtgaggagcagaagtgcgaagaggaggtcttcccgctggccatgaactacctggaccgcttcctgtccctggagcccttgaagaagagccgcctgcagctgctgggggccacctgcatgttcgtggcctctaagatgaaggagaccattcccttgactgccgagaagttgtgcatctacactgacaactctatccggcccgaggagctgctgcaaatggaactgcttctggtgaacaagctcaagtggaacctggccgccatgactccccacgatttcatcgaacacttcctctccaaaatgccagaggcggatgagaacaagcagaccatccgcaagcatgcacagacctttgtggccctctgtgccacagatgtgaagttcatttccaacccaccctccatggtagctgctgggagcgtggtggctgcgatgcaaggcctgaacctgggcagccccaacaacttcctctcctgctaccgcacaacgcactttctttccagagtcatcaagtgtgacccggactgcctccgtgcctgccaggaacagattgaagcccttctggagtcaagcctgcgccaggcccagcagaacgtcgaccccaaggccactgaggaggagggggaagtggaggaagaggctggtctggcctgcacgcccaccgacgtgcgagatgtggacatctga

SEQ ID NO: 104 - DNA, 564 bp, Mus musculus
acagaatacaagcttgtggtggtgggcgctggaggcgtgggaaagagtgccctgaccatccagctgatccagaaccactttgtggacgagtatgatcccactatagaggactcctaccggaaacaggtggtcattgatggggagacatgtctactggacatcttagacacagcaggtcaagaagagtatagtgccatgcgggaccagtacatgcgcacaggggagggcttcctctgtgtatttgccatcaacaacaccaagtccttcgaggacatccatcagtacagggagcagatcaagcgggtgaaagattcagatgatgtgccaatggtgctggtgggcaacaagtgtgacctggctgctcgcactgttgagtctcggcaggcccaggaccttgctcgcagctatggcatcccctacattgaaacatcagccaagacccggcagggcgtggaggatgccttctatacactagtccgtgagattcggcagcataaattgcggaaactgaacccacccgatgagagtggtcctggctgcatgagctgcaaatgtgtgctgtcc

SEQ ID NO: 105 - DNA, 3216 bp, Homo sapiens
atgccagcaattatgacaatgttagcagaccatgcagctcgtcagctgcttgatttcagccaaaaactggatatcaacttattagataatgtggtgaattgcttataccatggagaaggagcccagcaaagaatggctcaagaagtactgacacatttaaaggagcatcctgatgcttggacaagagtcgacacaattttggaattttctcagaatatgaatacgaaatactatggactacaaattttggaaaatgtgataaaaacaaggtggaagattcttccaaggaaccagtgcgaaggaataaaaaaatacgttgttggcctcattatcaagacgtcatctgacccaacttgtgtagagaaagaaaaggtgtatatcggaaaattaaatatgatccttgttcagatactgaaacaagaatggcccaaacattggccaacttttatcagtgatattgttggagcaagtaggaccagcgaaagtctctgtcaaaataatatggtgattcttaaactcttgagtgaagaagtatttgatttctctagtggacagataacccaagtcaaatctaagcatttaaaagacagcatgtgcaatgaattctcacagatatttcaactgtgtcagtttgtaatggaaaattctcaaaatgctccacttgtacatgcaaccttggaaacattgctcagatttctgaactggattcccctgggatatatttttgagaccaaattaatcagcacattgatttataagttcctgaatgttccaatgtttcgaaatgtctctctgaagtgcctcactgagattgctggtgtgagtgtaagccaatatgaagaacaatttgtaacactatttactctgacaatgatgcaactaaagcagatgcttcctttaaataccaatattcgacttgcgtactcaaatggaaaagatgatgaacagaacttcattcaaaatctcagtttgtttctctgcacctttcttaaggaacatgatcaacttatagaaaaaagattaaatctcagggaaactcttatggaggcccttcattatatgttgttggtatctgaagtagaagaaactgaaatctttaaaatttgtcttgaatactggaatcatttggctgctgaactctatagagagagtccattctctacatctgcctctccgttgctttctggaagtcaacattttgatgttcctcccaggagacagctatatttgcccatgttattcaaggtccgtttattaatggttagtcgaatggctaaaccagaggaagtattggttgtagagaatgatcaaggagaagttgtgagagaattcatgaaggatacagattccataaatttgtataagaatatgagggaaacattggtttatcttactcatctggattatgtagatacagaaagaataatgacagagaagcttcacaatcaagtgaatggtacagagtggtcatggaaaaatttgaatacattgtgttgggcaataggctccattagtggagcaatgcatgaagaggacgaaaaacgatttcttgttactgttataaaggatctattaggattatgtgaacagaaaagaggcaaagataataaagctattattgcatcaaatatcatgtacatagtaggtcaatacccacgttttttgagagctcactggaaatttctgaagactgtagttaacaagctgttcgaattcatgcatgagacccatgatggagtccaggatatggcttgtgatactttcattaaaatagcccaaaaatgccgcaggcatttcgttcaggttcaggttggagaagtgatgccatttattgatgaaattttgaacaacattaacactattatttgtgatcttcagcctcaacaggttcatacgttttatgaagctgtggggtacatgattggtgcacaaacagatcaaacagtacaagaacacttgatagaaaagtacatgttactccctaatcaagtgtgggatagtataatccagcaggcaaccaaaaatgtggatatactgaaagatcctgaaacagtcaagcagcttggtagcattttgaaaacaaatgtgagagcctgcaaagctgttggacacccctttgtaattcagcttggaagaatttatttagatatgcttaatgtatacaagtgcctcagtgaaaatatttctgcagctatccaagctaatggtgaaatggttacaaagcaaccattgattagaagtatgcgaactgtaaaaagggaaactttaaagttaatatctggttgggtgagccgatccaatgatccacagatggtcgctgaaaattttgttccccctctgttggatgcagttctcattgattatcagagaaatgtcccagctgctagagaaccagaagtgcttagtactatggccataattgtcaacaagttagggggacatataacagctgaaatacctcaaatatttgatgctgtttttgaatgcacattgaatatgataaataaggactttgaagaatatcctgaacatagaacgaactttttcttactacttcaggctgtcaattctcattgtttcccagcattccttgctattccacctacacagtttaaacttgttttggattccatcatttgggctttcaaacatactatgaggaatgtcgcagatacgggcttacagatactttttacactcttacaaaatgttgcacaagaagaagctgcagctcagagtttttatcaaacttatttttgtgatattctccagcatatcttttctgttgtgacagacacttcacatactgctggtttaacaatgcatgcatcaattcttgcatatatgtttaatttggttgaagaaggaaaaataagtacatcattaaatcctggaaatccagttaacaaccaaatctttcttcaggaatatgtggctaatctccttaagtcggccttccctcacctacaagatgctcaagtaaagctctttgtgacagggcttttcagcttaaatcaagatattcctgctttcaaggaacatttaagagatttcctagttcaaataaaggaatttgcaggtgaagacacttctgatttgtttttggaagagagagaaatagccctacggcaggctgatgaagagaaacataaacgtcaaatgtctgtccctggcatctttaatccacatgagattccagaagaaatgtgtgattaa

SEQ ID NO: 106 - DNA, 24 bp, Artificial sequence
  derived from a Kozak sequence
gactacaaggacgacgatgacaag

SEQ ID NO: 107 - DNA, 27 bp, Artificial sequence
  HA tag
tacccctacgacgtgcccgactacgcc

SEQ ID NO: 108 - DNA, 75 bp, Artificial sequence
  Ntag
gactacaaggacgacgatgacaagctcgatggaggatacccctacgacgtgcccgactacgccggaggactcgag

SEQ ID NO: 109 - DNA, 42 bp, Artificial sequence
  V5 tag
ggcaaaccgattccgaacccgctgctgggcctggatagcacc

SEQ ID NO: 110 - DNA, 30 bp, Artificial sequence
  Myc Tag
gaacagaaactgattagcgaagaggatctg

SEQ ID NO: 111 - DNA, 81 bp, Artificial sequence
  Ctag
gcggccgctggaggagactacaaggacgacgatgacaagtcggccgctggaggatacccctacgacgtgcccgactacgcc

SEQ ID NO: 112 - DNA, 3240 bp, Artificial sequence
  Myc - Xpo
gaacagaaactgattagcgaagaggatctgccagcaattatgacaatgttagcagaccatgcagctcgtcagctgcttgatttcagccaaaaactggatatcaacttattagataatgtggtgaattgcttataccatggagaaggagcccagcaaagaatggctcaagaagtactgacacatttaaaggagcatcctgatgcttggacaagagtcgacacaattttggaattttctcagaatatgaatacgaaatactatggactacaaattttggaaaatgtgataaaaacaaggtggaagattcttccaaggaaccagtgcgaaggaataaaaaaatacgttgttggcctcattatcaagacgtcatctgacccaacttgtgtagagaaagaaaaggtgtatatcggaaaattaaatatgatccttgttcagatactgaaacaagaatggcccaaacattggccaacttttatcagtgatattgttggagcaagtaggaccagcgaaagtctctgtcaaaataatatggtgattcttaaactcttgagtgaagaagtatttgatttctctagtggacagataacccaagtcaaatctaagcatttaaaagacagcatgtgcaatgaattctcacagatatttcaactgtgtcagtttgtaatggaaaattctcaaaatgctccacttgtacatgcaaccttggaaacattgctcagatttctgaactggattcccctgggatatatttttgagaccaaattaatcagcacattgatttataagttcctgaatgttccaatgtttcgaaatgtctctctgaagtgcctcactgagattgctggtgtgagtgtaagccaatatgaagaacaatttgtaacactatttactctgacaatgatgcaactaaagcagatgcttcctttaaataccaatattcgacttgcgtactcaaatggaaaagatgatgaacagaacttcattcaaaatctcagtttgtttctctgcacctttcttaaggaacatgatcaacttatagaaaaaagattaaatctcagggaaactcttatggaggcccttcattatatgttgttggtatctgaagtagaagaaactgaaatctttaaaatttgtcttgaatactggaatcatttggctgctgaactctatagagagagtccattctctacatctgcctctccgttgctttctggaagtcaacattttgatgttcctcccaggagacagctatatttgcccatgttattcaaggtccgtttattaatggttagtcgaatggctaaaccagaggaagtattggttgtagagaatgatcaaggagaagttgtgagagaattcatgaaggatacagattccataaatttgtataagaatatgagggaaacattggtttatcttactcatctggattatgtagatacagaaagaataatgacagagaagcttcacaatcaagtgaatggtacagagtggtcatggaaaaatttgaatacattgtgttgggcaataggctccattagtggagcaatgcatgaagaggacgaaaaacgatttcttgttactgttataaaggatctattaggattatgtgaacagaaaagaggcaaagataataaagctattattgcatcaaatatcatgtacatagtaggtcaatacccacgttttttgagagctcactggaaatttctgaagactgtagttaacaagctgttcgaattcatgcatgagacccatgatggagtccaggatatggcttgtgatactttcattaaaatagcccaaaaatgccgcaggcatttcgttcaggttcaggttggagaagtgatgccatttattgatgaaattttgaacaacattaacactattatttgtgatcttcagcctcaacaggttcatacgttttatgaagctgtggggtacatgattggtgcacaaacagatcaaacagtacaagaacacttgatagaaaagtacatgttactccctaatcaagtgtgggatagtataatccagcaggcaaccaaaaatgtggatatactgaaagatcctgaaacagtcaagcagcttggtagcattttgaaaacaaatgtgagagcctgcaaagctgttggacacccctttgtaattcagcttggaagaatttatttagatatgcttaatgtatacaagtgcctcagtgaaaatatttctgcagctatccaagctaatggtgaaatggttacaaagcaaccattgattagaagtatgcgaactgtaaaaagggaaactttaaagttaatatctggttgggtgagccgatccaatgatccacagatggtcgctgaaaattttgttccccctctgttggatgcagttctcattgattatcagagaaatgtcccagctgctagagaaccagaagtgcttagtactatggccataattgtcaacaagttagggggacatataacagctgaaatacctcaaatatttgatgctgtttttgaatgcacattgaatatgataaataaggactttgaagaatatcctgaacatagaacgaactttttcttactacttcaggctgtcaattctcattgtttcccagcattccttgctattccacctacacagtttaaacttgttttggattccatcatttgggctttcaaacatactatgaggaatgtcgcagatacgggcttacagatactttttacactcttacaaaatgttgcacaagaagaagctgcagctcagagtttttatcaaacttatttttgtgatattctccagcatatcttttctgttgtgacagacacttcacatactgctggtttaacaatgcatgcatcaattcttgcatatatgtttaatttggttgaagaaggaaaaataagtacatcattaaatcctggaaatccagttaacaaccaaatctttcttcaggaatatgtggctaatctccttaagtcggccttccctcacctacaagatgctcaagtaaagctctttgtgacagggcttttcagcttaaatcaagatattcctgctttcaaggaacatttaagagatttcctagttcaaataaaggaatttgcaggtgaagacacttctgatttgtttttggaagagagagaaatagccctacggcaggctgatgaagagaaacataaacgtcaaatgtctgtccctggcatctttaatccacatgagattccagaagaaatgtgtgat

SEQ ID NO: 113 - DNA, 3252 bp, Artificial sequence
  XPO V5
ccagcaattatgacaatgttagcagaccatgcagctcgtcagctgcttgatttcagccaaaaactggatatcaacttattagataatgtggtgaattgcttataccatggagaaggagcccagcaaagaatggctcaagaagtactgacacatttaaaggagcatcctgatgcttggacaagagtcgacacaattttggaattttctcagaatatgaatacgaaatactatggactacaaattttggaaaatgtgataaaaacaaggtggaagattcttccaaggaaccagtgcgaaggaataaaaaaatacgttgttggcctcattatcaagacgtcatctgacccaacttgtgtagagaaagaaaaggtgtatatcggaaaattaaatatgatccttgttcagatactgaaacaagaatggcccaaacattggccaacttttatcagtgatattgttggagcaagtaggaccagcgaaagtctctgtcaaaataatatggtgattcttaaactcttgagtgaagaagtatttgatttctctagtggacagataacccaagtcaaatctaagcatttaaaagacagcatgtgcaatgaattctcacagatatttcaactgtgtcagtttgtaatggaaaattctcaaaatgctccacttgtacatgcaaccttggaaacattgctcagatttctgaactggattcccctgggatatatttttgagaccaaattaatcagcacattgatttataagttcctgaatgttccaatgtttcgaaatgtctctctgaagtgcctcactgagattgctggtgtgagtgtaagccaatatgaagaacaatttgtaacactatttactctgacaatgatgcaactaaagcagatgcttcctttaaataccaatattcgacttgcgtactcaaatggaaaagatgatgaacagaacttcattcaaaatctcagtttgtttctctgcacctttcttaaggaacatgatcaacttatagaaaaaagattaaatctcagggaaactcttatggaggcccttcattatatgttgttggtatctgaagtagaagaaactgaaatctttaaaatttgtcttgaatactggaatcatttggctgctgaactctatagagagagtccattctctacatctgcctctccgttgctttctggaagtcaacattttgatgttcctcccaggagacagctatatttgcccatgttattcaaggtccgtttattaatggttagtcgaatggctaaaccagaggaagtattggttgtagagaatgatcaaggagaagttgtgagagaattcatgaaggatacagattccataaatttgtataagaatatgagggaaacattggtttatcttactcatctggattatgtagatacagaaagaataatgacagagaagcttcacaatcaagtgaatggtacagagtggtcatggaaaaatttgaatacattgtgttgggcaataggctccattagtggagcaatgcatgaagaggacgaaaaacgatttcttgttactgttataaaggatctattaggattatgtgaacagaaaagaggcaaagataataaagctattattgcatcaaatatcatgtacatagtaggtcaatacccacgttttttgagagctcactggaaatttctgaagactgtagttaacaagctgttcgaattcatgcatgagacccatgatggagtccaggatatggcttgtgatactttcattaaaatagcccaaaaatgccgcaggcatttcgttcaggttcaggttggagaagtgatgccatttattgatgaaattttgaacaacattaacactattatttgtgatcttcagcctcaacaggttcatacgttttatgaagctgtggggtacatgattggtgcacaaacagatcaaacagtacaagaacacttgatagaaaagtacatgttactccctaatcaagtgtgggatagtataatccagcaggcaaccaaaaatgtggatatactgaaagatcctgaaacagtcaagcagcttggtagcattttgaaaacaaatgtgagagcctgcaaagctgttggacacccctttgtaattcagcttggaagaatttatttagatatgcttaatgtatacaagtgcctcagtgaaaatatttctgcagctatccaagctaatggtgaaatggttacaaagcaaccattgattagaagtatgcgaactgtaaaaagggaaactttaaagttaatatctggttgggtgagccgatccaatgatccacagatggtcgctgaaaattttgttccccctctgttggatgcagttctcattgattatcagagaaatgtcccagctgctagagaaccagaagtgcttagtactatggccataattgtcaacaagttagggggacatataacagctgaaatacctcaaatatttgatgctgtttttgaatgcacattgaatatgataaataaggactttgaagaatatcctgaacatagaacgaactttttcttactacttcaggctgtcaattctcattgtttcccagcattccttgctattccacctacacagtttaaacttgttttggattccatcatttgggctttcaaacatactatgaggaatgtcgcagatacgggcttacagatactttttacactcttacaaaatgttgcacaagaagaagctgcagctcagagtttttatcaaacttatttttgtgatattctccagcatatcttttctgttgtgacagacacttcacatactgctggtttaacaatgcatgcatcaattcttgcatatatgtttaatttggttgaagaaggaaaaataagtacatcattaaatcctggaaatccagttaacaaccaaatctttcttcaggaatatgtggctaatctccttaagtcggccttccctcacctacaagatgctcaagtaaagctctttgtgacagggcttttcagcttaaatcaagatattcctgctttcaaggaacatttaagagatttcctagttcaaataaaggaatttgcaggtgaagacacttctgatttgtttttggaagagagagaaatagccctacggcaggctgatgaagagaaacataaacgtcaaatgtctgtccctggcatctttaatccacatgagattccagaagaaatgtgtgatggcaaaccgattccgaacccgctgctgggcctggatagcacc

SEQ ID NO: 114 - DNA, 3282 bp, Artificial sequence
  MYC  XPO V5
gaacagaaactgattagcgaagaggatctgccagcaattatgacaatgttagcagaccatgcagctcgtcagctgcttgatttcagccaaaaactggatatcaacttattagataatgtggtgaattgcttataccatggagaaggagcccagcaaagaatggctcaagaagtactgacacatttaaaggagcatcctgatgcttggacaagagtcgacacaattttggaattttctcagaatatgaatacgaaatactatggactacaaattttggaaaatgtgataaaaacaaggtggaagattcttccaaggaaccagtgcgaaggaataaaaaaatacgttgttggcctcattatcaagacgtcatctgacccaacttgtgtagagaaagaaaaggtgtatatcggaaaattaaatatgatccttgttcagatactgaaacaagaatggcccaaacattggccaacttttatcagtgatattgttggagcaagtaggaccagcgaaagtctctgtcaaaataatatggtgattcttaaactcttgagtgaagaagtatttgatttctctagtggacagataacccaagtcaaatctaagcatttaaaagacagcatgtgcaatgaattctcacagatatttcaactgtgtcagtttgtaatggaaaattctcaaaatgctccacttgtacatgcaaccttggaaacattgctcagatttctgaactggattcccctgggatatatttttgagaccaaattaatcagcacattgatttataagttcctgaatgttccaatgtttcgaaatgtctctctgaagtgcctcactgagattgctggtgtgagtgtaagccaatatgaagaacaatttgtaacactatttactctgacaatgatgcaactaaagcagatgcttcctttaaataccaatattcgacttgcgtactcaaatggaaaagatgatgaacagaacttcattcaaaatctcagtttgtttctctgcacctttcttaaggaacatgatcaacttatagaaaaaagattaaatctcagggaaactcttatggaggcccttcattatatgttgttggtatctgaagtagaagaaactgaaatctttaaaatttgtcttgaatactggaatcatttggctgctgaactctatagagagagtccattctctacatctgcctctccgttgctttctggaagtcaacattttgatgttcctcccaggagacagctatatttgcccatgttattcaaggtccgtttattaatggttagtcgaatggctaaaccagaggaagtattggttgtagagaatgatcaaggagaagttgtgagagaattcatgaaggatacagattccataaatttgtataagaatatgagggaaacattggtttatcttactcatctggattatgtagatacagaaagaataatgacagagaagcttcacaatcaagtgaatggtacagagtggtcatggaaaaatttgaatacattgtgttgggcaataggctccattagtggagcaatgcatgaagaggacgaaaaacgatttcttgttactgttataaaggatctattaggattatgtgaacagaaaagaggcaaagataataaagctattattgcatcaaatatcatgtacatagtaggtcaatacccacgttttttgagagctcactggaaatttctgaagactgtagttaacaagctgttcgaattcatgcatgagacccatgatggagtccaggatatggcttgtgatactttcattaaaatagcccaaaaatgccgcaggcatttcgttcaggttcaggttggagaagtgatgccatttattgatgaaattttgaacaacattaacactattatttgtgatcttcagcctcaacaggttcatacgttttatgaagctgtggggtacatgattggtgcacaaacagatcaaacagtacaagaacacttgatagaaaagtacatgttactccctaatcaagtgtgggatagtataatccagcaggcaaccaaaaatgtggatatactgaaagatcctgaaacagtcaagcagcttggtagcattttgaaaacaaatgtgagagcctgcaaagctgttggacacccctttgtaattcagcttggaagaatttatttagatatgcttaatgtatacaagtgcctcagtgaaaatatttctgcagctatccaagctaatggtgaaatggttacaaagcaaccattgattagaagtatgcgaactgtaaaaagggaaactttaaagttaatatctggttgggtgagccgatccaatgatccacagatggtcgctgaaaattttgttccccctctgttggatgcagttctcattgattatcagagaaatgtcccagctgctagagaaccagaagtgcttagtactatggccataattgtcaacaagttagggggacatataacagctgaaatacctcaaatatttgatgctgtttttgaatgcacattgaatatgataaataaggactttgaagaatatcctgaacatagaacgaactttttcttactacttcaggctgtcaattctcattgtttcccagcattccttgctattccacctacacagtttaaacttgttttggattccatcatttgggctttcaaacatactatgaggaatgtcgcagatacgggcttacagatactttttacactcttacaaaatgttgcacaagaagaagctgcagctcagagtttttatcaaacttatttttgtgatattctccagcatatcttttctgttgtgacagacacttcacatactgctggtttaacaatgcatgcatcaattcttgcatatatgtttaatttggttgaagaaggaaaaataagtacatcattaaatcctggaaatccagttaacaaccaaatctttcttcaggaatatgtggctaatctccttaagtcggccttccctcacctacaagatgctcaagtaaagctctttgtgacagggcttttcagcttaaatcaagatattcctgctttcaaggaacatttaagagatttcctagttcaaataaaggaatttgcaggtgaagacacttctgatttgtttttggaagagagagaaatagccctacggcaggctgatgaagagaaacataaacgtcaaatgtctgtccctggcatctttaatccacatgagattccagaagaaatgtgtgatggcaaaccgattccgaacccgctgctgggcctggatagcacc

SEQ ID NO: 115 - DNA, 909 bp, Artificial sequence
  HA CD1
tacccctacgacgtgcccgactacgccgaacaccagctcctgtgctgcgaagtggagaccatccgccgcgcgtaccctgacaccaatctcctcaacgaccgggtgctgcgagccatgctcaagacggaggagacctgtgcgccctccgtatcttacttcaagtgcgtgcagaaggagattgtgccatccatgcggaaaatcgtggccacctggatgctggaggtctgtgaggagcagaagtgcgaagaggaggtcttcccgctggccatgaactacctggaccgcttcctgtccctggagcccttgaagaagagccgcctgcagctgctgggggccacctgcatgttcgtggcctctaagatgaaggagaccattcccttgactgccgagaagttgtgcatctacactgacaactctatccggcccgaggagctgctgcaaatggaactgcttctggtgaacaagctcaagtggaacctggccgccatgactccccacgatttcatcgaacacttcctctccaaaatgccagaggcggatgagaacaagcagaccatccgcaagcatgcacagacctttgtggccctctgtgccacagatgtgaagttcatttccaacccaccctccatggtagctgctgggagcgtggtggctgcgatgcaaggcctgaacctgggcagccccaacaacttcctctcctgctaccgcacaacgcactttctttccagagtcatcaagtgtgacccggactgcctccgtgcctgccaggaacagattgaagcccttctggagtcaagcctgcgccaggcccagcagaacgtcgaccccaaggccactgaggaggagggggaagtggaggaagaggctggtctggcctgcacgcccaccgacgtgcgagatgtggacatc

SEQ ID NO: 116 - DNA, 624 bp, Artificial sequence
  hybrid nucleic acid molecule
gccaccatgtacccctacgacgtgcccgactacgccacagaatacaagcttgtggtggtgggcgctggaggcgtgggaaagagtgccctgaccatccagctgatccagaaccactttgtggacgagtatgatcccactatagaggactcctaccggaaacaggtggtcattgatggggagacatgtctactggacatcttagacacagcaggtcaagaagagtatagtgccatgcgggaccagtacatgcgcacaggggagggcttcctctgtgtatttgccatcaacaacaccaagtccttcgaggacatccatcagtacagggagcagatcaagcgggtgaaagattcagatgatgtgccaatggtgctggtgggcaacaagtgtgacctggctgctcgcactgttgagtctcggcaggcccaggaccttgctcgcagctatggcatcccctacattgaaacatcagccaagacccggcagggcgtggaggatgccttctatacactagtccgtgagattcggcagcataaattgcggaaactgaacccacccgatgagagtggtcctggctgcatgagctgcaaatgtgtgctgtccgactacaaggacgacgatgacaag

SEQ ID NO: 117 - DNA, 624 bp, Artificial sequence
  hybrid nucleic acid molecule
gccaccatgtacccctacgacgtgcccgactacgccacagaatacaagcttgtggtggtgggcgctgtaggcgtgggaaagagtgccctgaccatccagctgatccagaaccactttgtggacgagtatgatcccactatagaggactcctaccggaaacaggtggtcattgatggggagacatgtctactggacatcttagacacagcaggtcaagaagagtatagtgccatgcgggaccagtacatgcgcacaggggagggcttcctctgtgtatttgccatcaacaacaccaagtccttcgaggacatccatcagtacagggagcagatcaagcgggtgaaagattcagatgatgtgccaatggtgctggtgggcaacaagtgtgacctggctgctcgcactgttgagtctcggcaggcccaggaccttgctcgcagctatggcatcccctacattgaaacatcagccaagacccggcagggcgtggaggatgccttctatacactagtccgtgagattcggcagcataaattgcggaaactgaacccacccgatgagagtggtcctggctgcatgagctgcaaatgtgtgctgtccgactacaaggacgacgatgacaag

SEQ ID NO: 118 - DNA, 974 bp, Artificial sequence
  hybrid nucleic acid molecule
cgcgccatatggaacaccagctcctgtgctgcgaagtggagaccatccgccgcgcgtaccctgacaccaatctcctcaacgaccgggtgctgcgagccatgctcaagacggaggagacctgtgcgccctccgtatcttacttcaagtgcgtgcagaaggagattgtgccatccatgcggaaaatcgtggccacctggatgctggaggtctgtgaggagcagaagtgcgaagaggaggtcttcccgctggccatgaactacctggaccgcttcctgtccctggagcccttgaagaagagccgcctgcagctgctgggggccacctgcatgttcgtggcctctaagatgaaggagaccattcccttgactgccgagaagttgtgcatctacactgacaactctatccggcccgaggagctgctgcaaatggaactgcttctggtgaacaagctcaagtggaacctggccgccatgactccccacgatttcatcgaacacttcctctccaaaatgccagaggcggatgagaacaagcagaccatccgcaagcatgcacagacctttgtggccctctgtgccacagatgtgaagttcatttccaacccaccctccatggtagctgctgggagcgtggtggctgcgatgcaaggcctgaacctgggcagccccaacaacttcctctcctgctaccgcacaacgcactttctttccagagtcatcaagtgtgacccggactgcctccgtgcctgccaggaacagattgaagcccttctggagtcaagcctgcgccaggcccagcagaacgtcgaccccaaggccactgaggaggagggggaagtggaggaagaggctggtctggcctgcacgcccaccgacgtgcgagatgtggacatcgcggccgctggaggagactacaaggacgacgatgacaagtcggccgctggaggatacccctacgacgtgcccgactacgcc

SEQ ID NO: 119 - DNA, 893 bp, Artificial sequence
  hybrid nucleic acid molecule
cgcgccatatggaacaccagctcctgtgctgcgaagtggagaccatccgccgcgcgtaccctgacaccaatctcctcaacgaccgggtgctgcgagccatgctcaagacggaggagacctgtgcgccctccgtatcttacttcaagtgcgtgcagaaggagattgtgccatccatgcggaaaatcgtggccacctggatgctggaggtctgtgaggagcagaagtgcgaagaggaggtcttcccgctggccatgaactacctggaccgcttcctgtccctggagcccttgaagaagagccgcctgcagctgctgggggccacctgcatgttcgtggcctctaagatgaaggagaccattcccttgactgccgagaagttgtgcatctacactgacaactctatccggcccgaggagctgctgcaaatggaactgcttctggtgaacaagctcaagtggaacctggccgccatgactccccacgatttcatcgaacacttcctctccaaaatgccagaggcggatgagaacaagcagaccatccgcaagcatgcacagacctttgtggccctctgtgccacagatgtgaagttcatttccaacccaccctccatggtagctgctgggagcgtggtggctgcgatgcaaggcctgaacctgggcagccccaacaacttcctctcctgctaccgcacaacgcactttctttccagagtcatcaagtgtgacccggactgcctccgtgcctgccaggaacagattgaagcccttctggagtcaagcctgcgccaggcccagcagaacgtcgaccccaaggccactgaggaggagggggaagtggaggaagaggctggtctggcctgcacgcccaccgacgtgcgagatgtggacatc

SEQ ID NO: 120 - DNA, 968 bp, Artificial sequence
  hybrid nucleic acid molecule
cgcgccatatggactacaaggacgacgatgacaagctcgatggaggatacccctacgacgtgcccgactacgccggaggactcgaggaacaccagctcctgtgctgcgaagtggagaccatccgccgcgcgtaccctgacaccaatctcctcaacgaccgggtgctgcgagccatgctcaagacggaggagacctgtgcgccctccgtatcttacttcaagtgcgtgcagaaggagattgtgccatccatgcggaaaatcgtggccacctggatgctggaggtctgtgaggagcagaagtgcgaagaggaggtcttcccgctggccatgaactacctggaccgcttcctgtccctggagcccttgaagaagagccgcctgcagctgctgggggccacctgcatgttcgtggcctctaagatgaaggagaccattcccttgactgccgagaagttgtgcatctacactgacaactctatccggcccgaggagctgctgcaaatggaactgcttctggtgaacaagctcaagtggaacctggccgccatgactccccacgatttcatcgaacacttcctctccaaaatgccagaggcggatgagaacaagcagaccatccgcaagcatgcacagacctttgtggccctctgtgccacagatgtgaagttcatttccaacccaccctccatggtagctgctgggagcgtggtggctgcgatgcaaggcctgaacctgggcagccccaacaacttcctctcctgctaccgcacaacgcactttctttccagagtcatcaagtgtgacccggactgcctccgtgcctgccaggaacagattgaagcccttctggagtcaagcctgcgccaggcccagcagaacgtcgaccccaaggccactgaggaggagggggaagtggaggaagaggctggtctggcctgcacgcccaccgacgtgcgagatgtggacatc

SEQ ID NO: 121 - DNA, 966 bp, artificial sequence
  hybrid nucleic acid sequence
ccagccatgtacccctacgacgtgcccgactacgccctcgatggaggagactacaaggacgacgatgacaagggaggactcgaggaacaccagctcctgtgctgcgaagtggaaaccatccgccgcgcgtaccccgatgccaacctcctcaacgaccgggtgctgcgggccatgctgaaggcggaggagacctgcgcgccctcggtgtcctacttcaaatgtgtgcagaaggaggtcctgccgtccatgcggaagatcgtcgccacctggatgctggaggtctgcgaggaacagaagtgcgaggaggaggtcttcccgctggccatgaactacctggaccgcttcctgtcgctggagcccgtgaaaaagagccgcctgcagctgctgggggccacttgcatgttcgtggcctctaagatgaaggagaccatccccctgacggccgagaagctgtgcatctacaccgacaactccatccggcccgaggagctgctgcaaatggagctgctcctggtgaacaagctcaagtggaacctggccgcaatgaccccgcacgatttcattgaacacttcctctccaaaatgccagaggcggaggagaacaaacagatcatccgcaaacacgcgcagaccttcgttgccctctgtgccacagatgtgaagttcatttccaatccgccctccatggtggcagcggggagcgtggtggccgcagtgcaaggcctgaacctgaggagccccaacaacttcctgtcctactaccgcctcacacgcttcctctccagagtgatcaagtgtgacccggactgcctccgggcctgccaggagcagatcgaagccctgctggagtcaagcctgcgccaggcccagcagaacatggaccccaaggccgccgaggaggaggaagaggaggaggaggaggtggacctggcttgcacacccaccgacgtgcgggacgtggacatc

SEQ ID NO: 122 - DNA, 944 bp, artificial sequence
  hybrid nucleic acid sequence
ccagccatatggactacaaggacgacgatgacaaggaacaccagctcctgtgctgcgaagtggaaaccatccgccgcgcgtaccccgatgccaacctcctcaacgaccgggtgctgcgggccatgctgaaggcggaggagacctgcgcgccctcggtgtcctacttcaaatgtgtgcagaaggaggtcctgccgtccatgcggaagatcgtcgccacctggatgctggaggtctgcgaggaacagaagtgcgaggaggaggtcttcccgctggccatgaactacctggaccgcttcctgtcgctggagcccgtgaaaaagagccgcctgcagctgctgggggccacttgcatgttcgtggcctctaagatgaaggagaccatccccctgacggccgagaagctgtgcatctacaccgacaactccatccggcccgaggagctgctgcaaatggagctgctcctggtgaacaagctcaagtggaacctggccgcaatgaccccgcacgatttcattgaacacttcctctccaaaatgccagaggcggaggagaacaaacagatcatccgcaaacacgcgcagaccttcgttgccctctgtgccacagatgtgaagttcatttccaatccgccctccatggtggcagcggggagcgtggtggccgcagtgcaaggcctgaacctgaggagccccaacaacttcctgtcctactaccgcctcacacgcttcctctccagagtgatcaagtgtgacccggactgcctccgggcctgccaggagcagatcgaagccctgctggagtcaagcctgcgccaggcccagcagaacatggaccccaaggccgccgaggaggaggaagaggaggaggaggaggtggacctggcttgcacacccaccgacgtgcgggacgtggacatctacccctacgacgtgcccgactacgcc

SEQ ID NO: 123 - DNA, 968 bp, artificial sequence
  hybrid nucleic acid sequence
ccagccatatggactacaaggacgacgatgacaagctcgatggaggatacccctacgacgtgcccgactacgccggaggactcgaggaacaccagctcctgtgctgcgaagtggaaaccatccgccgcgcgtaccccgatgccaacctcctcaacgaccgggtgctgcgggccatgctgaaggcggaggagacctgcgcgccctcggtgtcctacttcaaatgtgtgcagaaggaggtcctgccgtccatgcggaagatcgtcgccacctggatgctggaggtctgcgaggaacagaagtgcgaggaggaggtcttcccgctggccatgaactacctggaccgcttcctgtcgctggagcccgtgaaaaagagccgcctgcagctgctgggggccacttgcatgttcgtggcctctaagatgaaggagaccatccccctgacggccgagaagctgtgcatctacaccgacaactccatccggcccgaggagctgctgcaaatggagctgctcctggtgaacaagctcaagtggaacctggccgcaatgaccccgcacgatttcattgaacacttcctctccaaaatgccagaggcggaggagaacaaacagatcatccgcaaacacgcgcagaccttcgttgccctctgtgccacagatgtgaagttcatttccaatccgccctccatggtggcagcggggagcgtggtggccgcagtgcaaggcctgaacctgaggagccccaacaacttcctgtcctactaccgcctcacacgcttcctctccagagtgatcaagtgtgacccggactgcctccgggcctgccaggagcagatcgaagccctgctggagtcaagcctgcgccaggcccagcagaacatggaccccaaggccgccgaggaggaggaagaggaggaggaggaggtggacctggcttgcacacccaccgacgtgcgggacgtggacatc

SEQ ID NO: 124 - DNA, 944 bp, artificial sequence
  hybrid nucleic acid sequence
gactacaaggacgacgatgacaaggccaccatatggaacaccagctcctgtgctgcgaagtggagaccatccgccgcgcgtaccctgacaccaatctcctcaacgaccgggtgctgcgagccatgctcaagacggaggagacctgtgcgccctccgtatcttacttcaagtgcgtgcagaaggagattgtgccatccatgcggaaaatcgtggccacctggatgctggaggtctgtgaggagcagaagtgcgaagaggaggtcttcccgctggccatgaactacctggaccgcttcctgtccctggagcccttgaagaagagccgcctgcagctgctgggggccacctgcatgttcgtggcctctaagatgaaggagaccattcccttgactgccgagaagttgtgcatctacactgacaactctatccggcccgaggagctgctgcaaatggaactgcttctggtgaacaagctcaagtggaacctggccgccatgactccccacgatttcatcgaacacttcctctccaaaatgccagaggcggatgagaacaagcagaccatccgcaagcatgcacagacctttgtggccctctgtgccacagatgtgaagttcatttccaacccaccctccatggtagctgctgggagcgtggtggctgcgatgcaaggcctgaacctgggcagccccaacaacttcctctcctgctaccgcacaacgcactttctttccagagtcatcaagtgtgacccggactgcctccgtgcctgccaggaacagattgaagcccttctggagtcaagcctgcgccaggcccagcagaacgtcgaccccaaggccactgaggaggagggggaagtggaggaagaggctggtctggcctgcacgcccaccgacgtgcgagatgtggacatctacccctacgacgtgcccgactacgcc

SEQ ID NO: 125 - DNA, 945 bp, artificial sequence
  hybrid nucleic acid sequence
gccaccatatggaacaccagctcctgtgctgcgaagtggagaccatccgccgcgcgtaccctgacaccaatctcctcaacgaccgggtgctgcgagccatgctcaagacggaggagacctgtgcgccctccgtatcttacttcaagtgcgtgcagaaggagattgtgccatccatgcggaaaatcgtggccacctggatgctggaggtctgtgaggagcagaagtgcgaagaggaggtcttcccgctggccatgaactacctggaccgcttcctgtccctggagcccttgaagaagagccgcctgcagctgctgggggccacctgcatgttcgtggcctctaagatgaaggagaccattcccttgactgccgagaagttgtgcatctacactgacaactctatccggcccgaggagctgctgcaaatggaactgcttctggtgaacaagctcaagtggaacctggccgccatgactccccacgatttcatcgaacacttcctctccaaaatgccagaggcggatgagaacaagcagaccatccgcaagcatgcacagacctttgtggccctctgtgccacagatgtgaagttcatttccaacccaccctccatggtagctgctgggagcgtggtggctgcgatgcaaggcctgaacctgggcagccccaacaacttcctctcctgctaccgcacaacgcactttctttccagagtcatcaagtgtgacccggactgcctccgtgcctgccaggaacagattgaagcccttctggagtcaagcctgcgccaggcccagcagaacgtcgaccccaaggccactgaggaggagggggaagtggaggaagaggctggtctggcctgcacgcccaccgacgtgcgagatgtggacatctacccacgacgtgcccgactacgcctgagactacaaggacgacgatgacaag

SEQ ID NO: 126 - DNA, 956 bp, artificial sequence
  hybrid nucleic acid sequence
gactacaaggacgacgatgacaagggaagagcgccagccatatggaacaccagctcctgtgctgcgaagtggaaaccatccgccgcgcgtaccccgatgccaacctcctcaacgaccgggtgctgcgggccatgctgaaggcggaggagacctgcgcgccctcggtgtcctacttcaaatgtgtgcagaaggaggtcctgccgtccatgcggaagatcgtcgccacctggatgctggaggtctgcgaggaacagaagtgcgaggaggaggtcttcccgctggccatgaactacctggaccgcttcctgtcgctggagcccgtgaaaaagagccgcctgcagctgctgggggccacttgcatgttcgtggcctctaagatgaaggagaccatccccctgacggccgagaagctgtgcatctacaccgacaactccatccggcccgaggagctgctgcaaatggagctgctcctggtgaacaagctcaagtggaacctggccgcaatgaccccgcacgatttcattgaacacttcctctccaaaatgccagaggcggaggagaacaaacagatcatccgcaaacacgcgcagaccttcgttgccctctgtgccacagatgtgaagttcatttccaatccgccctccatggtggcagcggggagcgtggtggccgcagtgcaaggcctgaacctgaggagccccaacaacttcctgtcctactaccgcctcacacgcttcctctccagagtgatcaagtgtgacccggactgcctccgggcctgccaggagcagatcgaagccctgctggagtcaagcctgcgccaggcccagcagaacatggaccccaaggccgccgaggaggaggaagaggaggaggaggaggtggacctggcttgcacacccaccgacgtgcgggacgtggacatctgatacccctacgacgtgcccgactacgcc

SEQ ID NO: 127 - DNA, 989 bp, artificial sequence
  hybrid nucleic acid sequence
gccaccatatggaacagaaactgattagcgaagaggatctggctgccactcgatatgaacccgtggctgaaattggtgtcggtgcctatgggacggtgtacaaagcccgagatccccacagtggccactttgtggccctcaagagtgtgagagttcctaatggaggagcagctggagggggccttcccgtcagcacagttcgtgaggtggccttgttaaggaggctggaggcctttgaacatcccaatgttgtacggctgatggatgtctgtgctacttcccgaactgatcgggacatcaaggtcaccctagtgtttgagcatatagaccaggacctgaggacatacctggacaaagcacctccaccgggcctgccggttgagaccattaaggatctaatgcgtcagtttctaagcggcctggattttcttcatgcaaactgcattgttcaccgggacctgaagccagagaacattctagtgacaagtaatgggaccgtcaagctggctgactttggcctagctagaatctacagctaccagatggccctcacgcctgtggtggttacgctctggtaccgagctcctgaagttcttctgcagtctacatacgcaacacccgtggacatgtggagcgttggctgtatctttgcagagatgttccgtcggaagcctctcttctgtggaaactctgaagccgaccagttggggaaaatctttgatctcattggattgcctccagaagacgactggcctcgagaggtatctctacctcgaggagcctttgcccccagagggcctcggccagtgcagtcagtggtgccagagatggaggagtctggagcgcagctgctactggaaatgctgacctttaacccacataagcgaatctctgccttccgagccctgcagcactcctacctgcacaaggaggaaagcgacgcagagggcaaaccgattccgaacccgctgctgggcctggatagcacc

SEQ ID NO: 128 - DNA, 629 bp, artificial sequence
  hybrid nucleic acid sequence
gactacaaggacgacgatgacaaggccaccatatgggaacagaatacaagcttgtggtggtgggcgctggaggcgtgggaaagagtgccctgaccatccagctgatccagaaccactttgtggacgagtatgatcccactatagaggactcctaccggaaacaggtggtcattgatggggagacatgtctactggacatcttagacacagcaggtcaagaagagtatagtgccatgcgggaccagtacatgcgcacaggggagggcttcctctgtgtatttgccatcaacaacaccaagtccttcgaggacatccatcagtacagggagcagatcaagcgggtgaaagattcagatgatgtgccaatggtgctggtgggcaacaagtgtgacctggctgctcgcactgttgagtctcggcaggcccaggaccttgctcgcagctatggcatcccctacattgaaacatcagccaagacccggcagggcgtggaggatgccttctatacactagtccgtgagattcggcagcataaattgcggaaactgaacccacccgatgagagtggtcctggctgcatgagctgcaaatgtgtgctgtcctacccctacgacgtgcccgactacgcc

SEQ ID NO: 129 - DNA, 5992 bp, artificial sequence
  vector
ctgcagcctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcatcgttctgtgttgtctctgtctgactgtgtttctgtatttgtctgaaaattagggccagactgttaccactcccttaagtttgaccttagatcactggaaagatgtcgagcggctcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcttccatccgcgccgtctctcccccttgaacctcctctttcgaccccgcctcaatcctccctttatccagccctcactccttctctaggcgccggccggatcccagtgtggtggtacgtaggaattcggtaccagatctctcgaggggcgcgccataacttcgtataatgtatgctatacgaagttattaggtactgccgggcctcttgcggggaattgaagttcctatactttctagagaataggaacttcggaataggacttcaattctaccgggtaggggaggcgcttttcccaaggcagtctggagcatgcgctttagcagccccgctggcacttggcgctacacaagtggcctctggcctcgcacacattccacatccaccggtagcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcgaaggtcctcccgaggcccggcattctcgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttctgacgagtcttctgaggggatccatccgtcctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatcgaagttcctatctttctagagaataggacttcggaataggaacttcggcgcgccgggatccctagaccgcggaagcttatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttccgacttgtggtctcgctgccttgggagggtctcctctgagtgattgactacccgtcagcgggggtcttcacatgcagcatgtatcaaaattaatttggttttttttcttaagtatttacattaaatggccatagttgcattaatgaatcggccaacgcgcggggagaggcggtttgcgtattggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagtttgcggccgcaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 130 - DNA, 5169 bp, artificial sequence
  vector
ctgcagcctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcatcgttctgtgttgtctctgtctgactgtgtttctgtatttgtctgaaaattagggccagactgttaccactcccttaagtttgaccttagatcactggaaagatgtcgagcggctcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcttccatccgcgccgtctctcccccttgaacctcctctttcgaccccgcctcaatcctccctttatccagccctcactccttctctaggcgccggccggatcccagtgtggtggtacgtaggaattcgccagcacagtggtcgaccctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgaccccatgcatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttccgacttgtggtctcgctgccttgggagggtctcctctgagtgattgactacccgtcagcgggggtcttcacatgcagcatgtatcaaaattaatttggttttttttcttaagtatttacattaaatggccatagttgcattaatgaatcggccaacgcgcggggagaggcggtttgcgtattggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagtttgcggccgcaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 131 - DNA, 6113 bp, artificial sequence
  vector
aattcgccagcacagtggtcgaccctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgaccccatgcatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattgggatcccagtgtggtggtacgtagg

SEQ ID NO: 132 - DNA, 32 bp, Artificial Sequence
  sense oligonucleotide hybridizing with a Kozak sequence
tggtacggcgccaccatatggactacaaggac

SEQ ID NO: 133 - DNA, 32 bp, Artificial Sequence
  antisense oligonucleotide hybridizing with Kozac sequence
gtccttgtagtccatatggtggcgccgtacca

SEQ ID NO: 134 - DNA, 75 bp, Artificial Sequence
  FLAG HA tag
actacaaggacgacgatgacaagctcgatggaggatacccctacgacgtgcccgactacgccggaggactcgagg

SEQ ID NO: 135 - DNA, 881 bp, Artificial Sequence
  truncated mouse CD1
aacaccagctcctgtgctgcgaagtggagaccatccgccgcgcgtaccctgacaccaatctcctcaacgaccgggtgctgcgagccatgctcaagacggaggagacctgtgcgccctccgtatcttacttcaagtgcgtgcagaaggagattgtgccatccatgcggaaaatcgtggccacctggatgctggaggtctgtgaggagcagaagtgcgaagaggaggtcttcccgctggccatgaactacctggaccgcttcctgtccctggagcccttgaagaagagccgcctgcagctgctgggggccacctgcatgttcgtggcctctaagatgaaggagaccattcccttgactgccgagaagttgtgcatctacactgacaactctatccggcccgaggagctgctgcaaatggaactgcttctggtgaacaagctcaagtggaacctggccgccatgactccccacgatttcatcgaacacttcctctccaaaatgccagaggcggatgagaacaagcagaccatccgcaagcatgcacagacctttgtggccctctgtgccacagatgtgaagttcatttccaacccaccctccatggtagctgctgggagcgtggtggctgcgatgcaaggcctgaacctgggcagccccaacaacttcctctcctgctaccgcacaacgcactttctttccagagtcatcaagtgtgacccggactgcctccgtgcctgccaggaacagattgaagcccttctggagtcaagcctgcgccaggcccagcagaacgtcgaccccaaggccactgaggaggagggggaagtggaggaagaggctggtctggcctgcacgcccaccgacgtgcgagatgtggacatc

SEQ ID NO: 136 - DNA, 966 bp, Artificial Sequence
  hybrid molecule comprising wild type Kozak sequence
cgcgccatggactacaaggacgacgatgacaagctcgatggaggatacccctacgacgtgcccgactacgccggaggactcgaggaacaccagctcctgtgctgcgaagtggagaccatccgccgcgcgtaccctgacaccaatctcctcaacgaccgggtgctgcgagccatgctcaagacggaggagacctgtgcgccctccgtatcttacttcaagtgcgtgcagaaggagattgtgccatccatgcggaaaatcgtggccacctggatgctggaggtctgtgaggagcagaagtgcgaagaggaggtcttcccgctggccatgaactacctggaccgcttcctgtccctggagcccttgaagaagagccgcctgcagctgctgggggccacctgcatgttcgtggcctctaagatgaaggagaccattcccttgactgccgagaagttgtgcatctacactgacaactctatccggcccgaggagctgctgcaaatggaactgcttctggtgaacaagctcaagtggaacctggccgccatgactccccacgatttcatcgaacacttcctctccaaaatgccagaggcggatgagaacaagcagaccatccgcaagcatgcacagacctttgtggccctctgtgccacagatgtgaagttcatttccaacccaccctccatggtagctgctgggagcgtggtggctgcgatgcaaggcctgaacctgggcagccccaacaacttcctctcctgctaccgcacaacgcactttctttccagagtcatcaagtgtgacccggactgcctccgtgcctgccaggaacagattgaagcccttctggagtcaagcctgcgccaggcccagcagaacgtcgaccccaaggccactgaggaggagggggaagtggaggaagaggctggtctggcctgcacgcccaccgacgtgcgagatgtggacatc

SEQ ID NO: 137 - RNA, 21 nt, Artificial sequence
  siRNA designed against SEQ ID NO 134
gagcuaccuccuauggggaug

SEQ ID NO: 138 - RNA, 21 nt, Artificial sequence
  siRNA designed against SEQ ID NO 134
augcugcacgggcugaugcgg

SEQ ID NO: 139 - RNA, 21 nt, Artificial sequence
  siRNA designed against SEQ ID NO 134
gggaugcugcacgggcugaug

SEQ ID NO: 140 - RNA, 21 nt, Artificial sequence
  siRNA designed against SEQ ID NO 134
auggggaugcugcacgggcug

SEQ ID NO: 141 - RNA, 21 nt, Artificial sequence
  siRNA designed against SEQ ID NO 134
uggggaugcugcacgggcuga

SEQ ID NO: 142 - RNA, 21 nt, Artificial sequence
  siRNA designed against SEQ ID NO 134
ggggaugcugcacgggcugau

SEQ ID NO: 143 - RNA, 21 nt, Artificial sequence
  siRNA designed against SEQ ID NO 134
ggaugcugcacgggcugaugc

SEQ ID NO: 144 - RNA, 21 nt, Artificial sequence
  siRNA designed against SEQ ID NO 134
gaugcugcacgggcugaugcg

SEQ ID NO: 145 - RNA, 21 nt, Artificial sequence
  siRNA designed against SEQ ID NO 134
agccggcgaccuccuaugggg

SEQ ID NO: 146 - RNA, 21 nt, Artificial sequence
  siRNA designed against SEQ ID NO 134
cugcugcuacuguucgagcua

SEQ ID NO: 147 - RNA, 21 nt, Artificial sequence
  siRNA designed against SEQ ID NO 134
uuccugcugcuacuguucgag

SEQ ID NO: 148 - RNA, 21 nt, Artificial sequence
  siRNA designed against SEQ ID NO 134
auguuccugcugcuacuguuc

SEQ ID NO: 149 - RNA, 21 nt, Artificial sequence
  siRNA designed against SEQ ID NO 134
cugauguuccugcugcuacug

SEQ ID NO: 150 - RNA, 21 nt, Artificial sequence
  siRNA designed against SEQ ID NO 134
cugauguuccugcugcuacug

SEQ ID NO: 151 - RNA, 21 nt, Artificial sequence
  siRNA designed against SEQ ID NO 134
ugauguuccugcugcuacugu

SEQ ID NO: 152 - RNA, 21 nt, Artificial sequence
  siRNA designed against SEQ ID NO 134
gauguuccugcugcuacuguu

SEQ ID NO: 153 - RNA, 21 nt, Artificial sequence
  siRNA designed against SEQ ID NO 134
accugauguuccugcugcuac

SEQ ID NO: 154 - RNA, 21 nt, Artificial sequence
  siRNA designed against SEQ ID NO 134
auguuccugcugcugcuauuc

SEQ ID NO: 155 - RNA, 21 nt, Artificial sequence
  siRNA designed against SEQ ID NO 134
cugcugcuacuguucagccgg

SEQ ID NO: 156 - RNA, 21 nt, Artificial sequence
  siRNA designed against SEQ ID NO 134
uuccugcugcuacuguucagc

SEQ ID NO: 157 - DNA, 32 bp, Artificial Sequence
  oligo sense for mutation G->T
  misc_feature (21)..(21): R = G or A
tggtacggcgccaccatgttractacaaggac

SEQ ID NO: 158 - DNA, 32 bp, Artificial Sequence
  oligo antisense for mutation G->T
  misc_feature (12)..(12): Y = C or T
gtccttgtagtyaacatggtggcgccgtacca

SEQ ID NO: 159 - DNA, 10 bp, Artificial Sequence
  sequence of the wild type Kozak region
ccatggcgcg

SEQ ID NO: 160 - DNA, 10 bp, Artificial Sequence
  sequence of the Kozak region with AT insertion
ccatatggcg

SEQ ID NO: 161 - DNA, 21 bp, Artificial Sequence
  sense siRNA scramble
uucuccgaacgugucacgutt

SEQ ID NO: 162 - DNA, 21 bp, Artificial Sequence
  antisense siRNA scramble
acgugacacauucggagaatt

(19) United States
(12) Patent Application Publication
(10) Pub. No.: US 2017/0298351 A1
(43) Pub. Date: Oct. 19, 2017

(54) Method For Screening Interfering Molecules

(71) Applicants: Centre National de la Recherche Scientifique (CNRS), Paris Cedex 16 (FR); Université de Montpellier, Montpellier (FR)

(72) Inventor: Frédéric Bienvenu, Saint-Clément-de-Rivière (FR)

(21) Appl. No.: 15/514,386

(22) PCT Filed: Sep. 28, 2015
(86) PCT No.: PCT/FR2015/052572

§ 371 Date: Mar. 24, 2017

(30) Foreign Application Priority Data
Sep. 26, 2014 (FR) 1459151

(51) Int. Cl. C12N 15/113; C12Q 1/68

(52) U.S. Cl. CPC C12N 15/113; C12Q 1/6876; C12Q 1/6897; C12N 2310/14; C12N 2320/11; C12Q 2600/178

(57) ABSTRACT

A method for screening interfering nucleic acids enhancing the expression or the activity of expressed gene sequences is described. The method includes a step of introducing, into a cell, a hybrid nucleic acid molecule having: a first non-coding sequence intended to initiate translation; a second sequence complementary to the interfering nucleic acids to be screened; and, optionally, a third nucleotide sequence encoding at least one pre-determined peptide, the first sequence being modified such that the translation level of the at least one peptide is reduced by at least 10%.

### FIELD OF THE INVENTION

The present invention relates to a method for screening interfering molecules.

### BACKGROUND OF THE INVENTION

The known phenomenon of RNA interference (RNAi) is based on the fact that small ribonucleic acid molecules can interact with messenger RNAs. A complex mechanism, controlled by numerous enzymes, leads to the degradation of the messenger RNAs, thereby inhibiting the expression of the genes encoding said messenger RNAs and consequently inhibiting the expression of the proteins resulting therefrom.

Among the small interfering RNAs, several species of RNA have been identified, especially micro-RNAs and hairpin RNAs, which are capable of inhibiting gene expression, and therefore the proteins which result therefrom, by similar mechanisms.

It is currently very widely accepted to test, for each of the genes of interest for which inhibition by RNA interference is sought, to individually test each interfering RNA for each of the genes considered. Such a process is long and costly, because it is necessary to verify that each interfering RNA considered does indeed exert its inhibitory effect on the expression of the target gene. Moreover, it is necessary to verify that each of the interfering RNAs considered to exert an appropriate inhibitory effect does not also exert a “parasitic” inhibitory effect by also blocking the expression of one or more genes other than that which is targeted initially.

U.S. Pat. No. 8,252,535 describes the use of an artificial sequence comprising a sequence to be targeted which is complementary to the sequence of a known interfering RNA. This method moreover makes it possible to simultaneously inhibit RNAs encoding different target genes. However, such a method remains imperfect, and especially does not make it possible to effectively screen interfering RNAs specific to a natural target.

Nucleic acid molecules having a positive effect on gene expression are also known from the prior art.

For example, Voutila et al., Molecular Therapy, Aug. 1, 2012 describes siRNAs used to activate the genes involved in cellular pluripotency. The application WO2006113246A2 describes sRNAs which activate gene expression, especially by binding to promoter regions. Nonetheless, these molecules have the effect of targeting regulatory sequences but do not specifically target the coding sequences of genes.

Thus, the aim of the invention is to overcome these drawbacks.

### SUMMARY OF THE INVENTION

One of the aims of the invention is to provide a method for screening interfering molecules having better sensitivity.

Another aim of the invention relates to a hybrid nucleic acid making it possible to carry out a method for screening interfering molecules, this method being more sensitive than those known from the prior art.

Yet another aim of the invention is to provide means making it possible to easily and effectively carry out the above-mentioned method.

Thus, the invention relates to a method for screening, especially in vitro, interfering nucleic acids increasing:
gene expression and/or
the activity of genes and/or of ribonucleic acids or RNAs transcribed from said genes,
said interfering nucleic acids having at least partial sequence complementarity with said genes or said RNAs, and
said method comprising a step of introducing, into a eukaryotic cell, especially capable of RNA interference, a hybrid nucleic acid molecule comprising:
a first non-coding sequence intended to initiate translation,
a second sequence at least partially complementary to the sequence of said interfering nucleic acids to be screened, and
a third nucleotide sequence encoding at least one determined peptide, said third sequence being under cis translational control of the first sequence,
said first sequence being modified, especially by substitution or deletion or addition of at least one nucleotide, such that the level of translation of said at least one peptide is reduced by at least 10% relative to the level of translation of said at least one peptide under control of said first sequence in its unmodified, especially optimal, version.

The invention is based on the surprising observation made by the inventors that it is possible to screen interfering molecules making it possible to increase gene expression when said molecules are selected by means of a nucleic acid molecule having a modified (non-optimal) translation initiation sequence.

Thus, the inventors have identified, for the first time, interfering RNAs having properties contrary to those widely described and accepted in the prior art, namely known expression inhibition properties of the RNA interference mechanisms.

In the invention, it is essential to have a nucleic acid molecule which comprises a first translation initiation sequence which is not natural, and which differs from said sequence such that it may be found in wild-type eukaryotes (that is to say not having a mutation at said translation initiation sequence).

In the invention, translation initiation sequence is intended to mean the sequence of nucleic acids present in the genes and in the messenger RNAs which result therefrom, and which surrounds the start codon ATG. This sequence is more commonly referred to as the Kozak sequence.

In the invention, “optimal” is intended to mean the maximum level of translation operating for a determined gene in a given cell type and under a given culture condition. The optimal level of translation is the maximum level of production of a protein by an RNA under the control of said translation initiation site, where the charging of the first amino acid imported by the initiator transfer RNA, methionine, takes place. The cell is understood to be incapable, in the natural physiological state, of producing more protein for a given RNA than its optimal level.

In the invention, “increasing gene expression” is intended to mean all modifications which have the consequence of obtaining an amount of protein, encoded by said gene, that is higher than the amount of protein obtained without modification. Thus, in the presence of an interfering nucleic acid according to the invention, the protein product of a gene (targeted by said interfering nucleic acid) will be more abundant than the protein product of the same gene in the absence of an interfering nucleic acid targeting said gene, or in the presence of an interfering nucleic acid targeting another gene. This definition of “increasing gene expression” is a conventional definition used by those skilled in the art.

Thus, even if the molecular mechanism which involves this protein increase is post-transcriptional (stabilization of RNA, increase in transcription) reference will still be made to increase in gene expression.

In the invention, “increase in the activity of genes and/or of ribonucleic acids or RNAs transcribed from said genes” corresponds, as indicated above, to one of the mechanisms leading to increase in gene expression, that is to say increase in the protein product encoded by said gene.

In the invention, interfering molecules are intended to mean nucleic acid molecules capable of regulating gene expression by RNA interference. One of the examples of interfering molecules covered by the invention are therefore small interfering RNAs (siRNAs), micro RNAs (miRNAs) or short hairpin RNAs (shRNAs).

siRNAs are small double-stranded RNAs containing 21 to 24 nucleotides. Small interfering RNAs in the double-stranded state are recognized in the cell cytoplasm by a protein complex referred to as the RISC complex (for RNA-induced silencing complex). The latter is activated by releasing the complementary strand of the RNA or the sense strand. This activated complex will recognize its target transcript, a messenger RNA, by complementarity of the nucleic bases. This system of recognition ensures the high specificity of this mechanism. Once the target is bound, the Argonaute protein, which is part of the RISC complex, can cleave the transcript at the recognition site. Ago can thus act as an endonuclease. The two pieces of the transcript cleaved by Ago will be rapidly degraded via their ends by exonucleases.

miRNAs are single-stranded RNAs capable of forming double-stranded structures by base pairing. During the formation of RISC, a double-stranded miRNA becomes a single-stranded miRNA. Only the specific strand of the messenger RNA which is the target for the miRNA is retained within the complex. The target mRNA is thus charged within the RISC complex. Two inhibition pathways are then possible; either the degradation of the target mRNA if the complex contains the protein Ago2 or the repression of the translation of this target mRNA if the complex contains the protein Ago1.

shRNAs are RNAs which adopt a stem-loop structure and which may be involved in the phenomenon of RNA interference. After incorporation by the RISC complex, the sense strand is degraded. The anti-sense strand directs the RISC complex to those mRNAs having a complementary sequence. The mechanism of degradation is thus similar to that adopted by siRNAs.

Within the context of the invention, the mechanism according to which some interfering molecules are capable of increasing gene expression has not yet been elucidated. Nonetheless, the inventors have shown, by means of the method according to the invention, that some molecules which are generally known in the prior art to inhibit the translation of messenger RNAs, or to destabilize messenger RNAs, are in fact capable of increasing the expression or the stability of said messenger RNAs.

In the invention, “said interfering nucleic acids having at least partial sequence complementarity with said gene or said RNA” means that the interfering nucleic acids to be screened are selected beforehand firstly so as to be at least partially complementary to the sequence of a gene, or to the messenger RNA that it encodes, such that the screening is specific. Moreover, those skilled in the art will understand that interfering nucleic acids cannot be entirely complementary to the sequence of the gene that they target insofar as they are smaller, in terms of number of nucleotides, than the targeted gene.

In the invention, hybrid nucleic acid molecule is intended to mean a hybrid nucleic acid molecule which is composed of at least two fragments of nucleic acids which are not adjacent in nature. This hydride molecule does not therefore exist in the natural state.

Said hybrid nucleic acid molecule, as mentioned above, comprises at least three sequences:
a first non-coding sequence intended to initiate translation, more commonly referred to as translation initiation sequence. This sequence is modified, that is to say that it has at least one nucleotide that is different relative to the same sequence observed in the general population,
a second sequence at least partially complementary to the sequence of said interfering nucleic acids to be screened, which corresponds to the target sequence of the interfering nucleic acid or acids, and
a third nucleotide sequence encoding at least one determined peptide, this peptide possibly corresponding to an immunogenic tag or else to a functional protein having an enzymatic activity or to a protein having luminescent or fluorescent properties.

In the invention, it is possible that the second sequence is contained within the third sequence. This means that the third sequence which encodes said at least one determined peptide comprises a portion of its sequence which is at least partially complementary to the sequence of said interfering nucleic acids to be screened. This may mean on the one hand that the second sequence corresponds to a portion of the third sequence, both encoding a portion of the determined peptide, or on the other hand that the determined peptide is “hybrid”, that is to say that it is encoded by a sequence in which an exogenous sequence (the second sequence) has been introduced. In this second case, the determined peptide will comprise a portion of its nucleic acid sequence which is not naturally included in the sequence of said determined peptide.

In the invention, it is also possible that the second and the third sequence are identical. This is especially the case when the sequence encoding the determined peptide is also entirely partially complementary, or completely complementary, to the interfering nucleic acids to be screened. This is especially the case for the sequence encoding the FLAG tag. If an interfering molecule increasing the expression of the tag is sought, the nucleic acid sequence encoding the FLAG tag is placed downstream of the first sequence and the hybrid nucleic acid molecule is then composed of three sequences (which in fact only represent two), the second and the third being totally the same, or identical.

In the hybrid nucleic acid molecule, there is a functional control of the third sequence by the first sequence, this control being exerted in cis, which means that the control occurs when the two sequences are borne by the same molecule.

The first sequence of the hybrid molecule is modified such that the translation of said at least one peptide is reduced by at least 10% relative to the level of translation of said at least one peptide under control of said first sequence in its unmodified, especially optimal, version. In other words, in the absence of interfering nucleic acids, a eukaryotic cell comprising a hybrid nucleic acid molecule not having a modified first sequence will express at least 10% more determined peptide (encoded by the third sequence) than the same eukaryotic cell comprising a hybrid nucleic acid molecule having said modified first sequence.

In order to enable an interaction between the hybrid nucleic acid molecule and the interfering nucleic acids to be screened, it is necessary for the interfering nucleic acid sequences and the second sequence of the hybrid nucleic acid molecule to be at least partially complementary, according to A-T/A-U and G-C base complementarity, well known to those skilled in the art.

In the invention, “at least partially complementary” is intended to mean the fact that the vast majority of nucleotides which compose the interfering nucleic acids to be screened are complementary to the nucleotides which define the second sequence of the hybrid nucleic acid molecule. In particular, it is advantageous for the nucleotides of the interfering nucleic acids to be screened and those which compose the second sequence of the hybrid nucleic acid molecule to be complementary to more than 90%, advantageously more than 95%, especially more than 99%, in particular 100%, or in other words for the two molecules to have less than 10%, advantageously less than 5%, especially less than 1%, in particular 0% mismatches. When the second sequence of the interfering nucleic acid molecule is composed of approximately 20 nucleotides, it is particularly advantageous for complementarity to be total.

Indeed, the invention relies on this surprising observation made by the inventors, according to which modification of the translation initiation sequence enables weaker expression of the determined peptide, which serves as marker, and hence makes it possible to more precisely visualize the variation in expression, especially the increase in expression, when the effectiveness of an interfering nucleic acid is tested.

The method according to the invention advantageously comprises the following steps:
A first step, which consists in transforming eukaryotic cells capable of RNA interference, by transfection techniques well known in the prior art, with a hybrid nucleic acid molecule. This may especially be electroporation, calcium phosphate transformation, lipofection, viral infection or else nucleofection. These examples of transformation of eukaryotic cells are given by way of indication and in no way limit the scope of the invention.
Once transformed, transiently or stably, the above-mentioned cells are ready to be used for the screening of interfering nucleic acids. It may be advantageous to have cells transformed stably (that is to say cells having integrated said hybrid nucleic acid molecule into their genome) in order to be able to always use the same transformed cell by simple cell culture.
In a second step, the cell or cells transformed in the preceding step are once again transformed by conventional means known to those skilled in the art, in order to introduce said interfering nucleic acids to be screened into said cells.
The cells transformed in this way with, on the one hand, the hybrid nucleic acid molecule and a population of an interfering nucleic acid to be screened, are cultured in a third step in order to be able to carry out the process of RNA interference for a determined length of time which those skilled in the art, with their general knowledge of RNA interference, can readily determine depending on the cell type used.
At the end of said determined length of time, the cells are then, in a fourth step, analyzed in order to measure the level of expression, that is to say the presence, the absence or the amount of said determined peptide encoded by said hybrid nucleic acid molecule. This presence, absence or amount of determined peptide is evaluated by comparison with the amount of said same determined peptide expressed by said cells transformed with said hybrid nucleic acid molecule, but which has not been transformed with interfering nucleic acids to be screened, or which has been transfected with interfering nucleic acids which do not have a target (that is to say complementary sequences) in the hybrid nucleic acid molecule.

If the amount of determined peptide in the cells transformed with an interfering nucleic acid is less than or equal to or greater by less than 10% than the amount of peptide in the cells which have not been transformed with any interfering nucleic acid or which have been transfected with interfering nucleic acids which do not have a target (that is to say complementary sequences) in the hybrid nucleic acid molecule, said interfering nucleic acid will not be retained. If, on the other hand, the amount of determined peptide in the cells transformed with an interfering nucleic acid is greater by at least 10% than the amount of peptide in the cells which have not been transformed with any interfering nucleic acid or which have been transfected with interfering nucleic acids which do not have a target (that is to say complementary sequences) in the hybrid nucleic acid molecule, then said interfering nucleic acid will be retained because it exerts an activating effect on the expression or the activity of the gene which it targets.

In order to measure the difference in the level of expression of at least 10%, it is possible to use conventional techniques of immunodetection capable of quantitatively detecting the light radiation emitted by chemiluminescent or fluorescent detection.

For example, it is possible to carry out immunolabeling of the peptide of interest by the Western blot technique, and to measure the amount thereof by chemiluminescence. Similarly, if the antibody directed against the peptide is coupled to a fluorescent marker, it is possible to measure the amount thereof by measuring the fluorescent radiation emitted after excitation at the appropriate wavelength.

When the peptide itself has autofluorescent properties, it is possible to measure the amount thereof directly from living cells, especially by flow cytometry.

It may also be advantageous for the hybrid nucleic acid molecule, by means of the third sequence, to encode at least two peptides which are able to carry out energy transfer between fluorescent molecules, or FRET.

In this specific case in which the third sequence encodes at least two peptides able to carry out FRET, it is possible to measure the amount of peptide expressed, and hence the effect of the interfering nucleic acid, directly on living cells transformed with the hybrid nucleic acid molecule, transformed or not transformed by an interfering nucleic acid, by measuring the fluorescence emission resulting from the energy transfer.

In summary, the method according to the invention is a method for screening interfering nucleic acids increasing:
gene expression and/or
the activity of genes and/or of ribonucleic acids transcribed from said genes,
said interfering nucleic acids having at least partial sequence complementarity with said gene or said RNA,
said method comprising
1—a step of introducing, into a eukaryotic cell, especially capable of RNA interference, a hybrid nucleic acid molecule comprising:
a first non-coding sequence intended to initiate translation,
a second sequence at least partially complementary to the sequence of said interfering nucleic acids to be screened, and
a third nucleotide sequence encoding at least one determined peptide, said third sequence being under cis translational control of the first sequence,
said first sequence being modified, by substitution, deletion or addition of at least one nucleotide, such that the level of translation of said at least one peptide is reduced by at least 10% relative to the level of translation of said at least one peptide under control of said first sequence in its unmodified, especially optimal, version,
2—a step of introducing an interfering nucleic acid into the eukaryotic cell obtained in the preceding step, and
3—a step of measuring the expression of said at least one peptide.

This method makes it possible to conclude that, if the level of expression of said at least one peptide is greater by 10% than the level of expression of said peptide expressed by a eukaryotic cell obtained in step 1, but which is not transformed with any interfering nucleic acid, or an interfering nucleic acid having no sequence complementarity with the second sequence of said hybrid nucleic acid molecule, the interfering nucleic acid which has enabled this increase in expression by more than 10% is an interfering nucleic acid of interest according to the invention.

In the opposite case, the interfering nucleic acid tested is not retained because it does not have properties of increasing the expression or activity of the targeted gene.

It should be noted that in the invention the hybrid nucleic acid molecule may either be a ribonucleic acid (RNA) or a single-stranded or double-stranded deoxyribonucleic acid (single-stranded DNA or double-stranded DNA). Advantageously, the hybrid nucleic acid molecule is a molecule of deoxyribonucleic acid.

Advantageously, the invention relates to the abovementioned method in which said first sequence is a Kozak sequence for downstream translation initiation either of an internal ribosome entry site or IRES, or of an RNA cap (5′CAP).

Translation initiation takes place by virtue of the presence of a Kozak sequence. Nonetheless, in order to enable translation initiation, it is necessary for ribosomes and all translation machinery to be “loaded” onto the messenger RNA. Such “loading” occurs either by means of an RNA cap (5′CAP) or by means of an internal ribosome entry sequence or IRES. These sequences (5′CAP/IRES) also have the ability to stabilize the messenger RNAs on which they are loaded, or even to export the messenger RNAs to their translation sites.

The RNA cap (5′CAP) is a modified nucleotide found at the 5′ end of messenger RNAs in eukaryotic cells. It is a post-transcriptional modification which is introduced by the successive action of several enzymes located in the nucleus. The cap is composed of a methylated guanosine at the position N7, linked to the first nucleotide of the transcribed messenger RNA by a 5′-5′ triphosphate bond.

IRESs enable the direct recruitment of ribosomes to the start codon, independently of the presence of the cap and of the scanning mechanism. IRESs are structured regions of the mRNA which interact directly with the ribosome or with the translation initiation factors.

In one advantageous embodiment, the invention relates to the method defined above, in which the nucleic acid molecule comprises said first sequence positioned upstream of, or in the 5′ position of, said third sequence.

In order to coordinate the cis regulation of the third sequence, it is advantageous for the first sequence of the hybrid nucleic acid molecule to be positioned upstream of the third sequence encoding the determined peptide. The first and third sequences may thus be directly connected, or adjacent, but may also be separated by another sequence, whether this is the second sequence or any other sequence.

In another advantageous embodiment, the invention relates to the above-mentioned method, in which said second sequence is positioned,
either in 5′ of said first sequence,
or in 3′ of said first sequence and in 5′ of said third sequence,
or in 3′ of said third sequence,
or contained within the third sequence.

The various possibilities of ordering of the sequences of the hybrid nucleic acid molecule are illustrated in FIG. 1.

In another advantageous embodiment, the invention relates to the method as defined above, in which said nucleic acid molecule is a molecule of deoxyribonucleic acid, especially double-stranded, optionally contained in a vector, or a molecule of ribonucleic acids, especially single-stranded.

As has been mentioned above, the hybrid nucleic acid molecule is introduced into a eukaryotic cell. This hybrid nucleic acid molecule may be introduced in different forms into said eukaryotic cell, namely:
in the form of a single-stranded ribonucleic acid or RNA, which will be used by the translation machinery of the eukaryotic cell to translate said at least one determined peptide encoded by said third sequence,
in the form of a deoxyribonucleic acid or DNA, in particular double-stranded, which will then have to be transcribed into RNA by the cell machinery of the eukaryotic cell, then translated; in this case it will be important for the hybrid nucleic acid molecule to comprise, aside from the three above-mentioned sequences, a sequence enabling transcription of an RNA,
in the form of a DNA vector comprising said hybrid nucleic acid sequence, this vector comprising means enabling the transcription of the hybrid nucleic acid molecule, especially a promoter and also a sequence enhancing transcription (enhancer). The vector may then be a circular vector and possibly comprise a eukaryotic or viral origin of replication in order that it may autonomously replicate itself, or a linearized vector in order to stimulate the integration thereof into the eukaryotic cell. It is moreover advantageous for said vector to comprise one or more sequences encoding proteins making it possible to select cells which have integrated said vector into their genome, for example, and without being limiting,
   sequences encoding peptides enabling resistance to certain antibiotics, such as puromycin, neomycin/G148, blasticidin, or else zeocin,
   sequences encoding autofluorescent proteins such as green fluorescent protein and derivatives thereof, or
   sequences encoding proteins exerting a negative selection on the cells which express it, such as for example thymidine kinase.

Even more advantageously the invention relates to the abovementioned method, in which said first sequence is a Kozak sequence represented, in its unmodified version, by the following sequence:
5′-ssmRccA(T/U)GG-3′ (SEQ ID NO: 1)
   in which R represents a purine, s represents G or C and m represents A/U or C.

This is the sequence SEQ ID NO: 1 which corresponds to the first sequence of the hybrid nucleic acid molecule which must be modified by suppression, deletion or insertion of at least one nucleotide.

In yet another embodiment, the invention relates to a method as defined above, in which
when the hybrid nucleic acid molecule is a molecule of deoxyribonucleic acids, especially double-stranded, said first sequence in its unmodified version is represented by the following sequence: 5′-ssmRccATGG-3′ (SEQ ID NO: 2), or
when the hybrid nucleic acid molecule is a molecule of ribonucleic acids, especially single-stranded, said first sequence in its unmodified version is represented by the following sequence: 5′-ssmRccAUGG-3′ (SEQ ID NO: 3).

Thus, the sequence SEQ ID NO: 2 covers the following different sequences:

| | |
|---|---|
| 5′-GGCGCCATGG-3′, | (SEQ ID NO: 4) |
| 5′-GGCACCATGG-3′, | (SEQ ID NO: 5) |
| 5′-GGAGCCATGG-3′, | (SEQ ID NO: 6) |
| 5′-GGAACCATGG-3′, | (SEQ ID NO: 7) |
| 5′-GCCGCCATGG-3′, | (SEQ ID NO: 8) |
| 5′-GCCACCATGG-3′, | (SEQ ID NO: 9) |
| 5′-GCAGCCATGG-3′, | (SEQ ID NO: 10) |
| 5′-GCAACCATGG-3′, | (SEQ ID NO: 11) |
| 5′-CGCGCCATGG-3′, | (SEQ ID NO: 12) |
| 5′-CGCACCATGG-3′, | (SEQ ID NO: 13) |
| 5′-CGAGCCATGG-3′, | (SEQ ID NO: 14) |
| 5′-CGAACCATGG-3′, | (SEQ ID NO: 15) |
| 5′-CCCGCCATGG-3′, | (SEQ ID NO: 16) |
| 5′-CCCACCATGG-3′, | (SEQ ID NO: 17) |
| 5′-CCAGCCATGG-3′, | (SEQ ID NO: 18) |
| and | |
| 5′-CCAACCATGG-3′. | (SEQ ID NO: 19) |

Similarly, the sequence SEQ ID NO: 3 covers the following different sequences:

| | |
|---|---|
| 5′-GGCGCCAUGG-3′, | (SEQ ID NO: 20) |
| 5′-GGCACCAUGG-3′, | (SEQ ID NO: 21) |
| 5′-GGAGCCAUGG-3′, | (SEQ ID NO: 22) |
| 5′-GGAACCAUGG-3′, | (SEQ ID NO: 23) |
| 5′-GCCGCCAUGG-3′, | (SEQ ID NO: 24) |
| 5′-GCCACCAUGG-3′, | (SEQ ID NO: 25) |
| 5′-GCAGCCAUGG-3′, | (SEQ ID NO: 26) |
| 5′-GCAACCAUGG-3′, | (SEQ ID NO: 27) |
| 5′-CGCGCCAUGG-3′, | (SEQ ID NO: 28) |
| 5′-CGCACCAUGG-3′, | (SEQ ID NO: 29) |
| 5′-CGAGCCAUGG-3′, | (SEQ ID NO: 30) |
| 5′-CGAACCAUGG-3′, | (SEQ ID NO: 31) |
| 5′-CCCGCCAUGG-3′, | (SEQ ID NO: 32) |
| 5′-CCCACCAUGG-3′, | (SEQ ID NO: 33) |
| 5′-CCAGCCAUGG-3′, | (SEQ ID NO: 34) |
| and | |
| 5′-CCAACCAUGG-3′. | (SEQ ID NO: 35) |

Advantageously, the invention relates to the abovementioned method, in which said first sequence is a Kozak sequence comprising or consisting of, in its modified version, one of the following sequences:

| | |
|---|---|
| 5′-SSMRCCA(T/U)Gt-3′, | (SEQ ID NO: 36) |
| or | |
| 5′-SSMRCCa(t/u)A(T/U)GG-3′. | (SEQ ID NO: 37) |

The inventors have observed, surprisingly, that the insertion of the doublet A(T/U) before the translation start codon of the Kozak sequence, or the substitution G->T after the translation start codon of the Kozak sequence, had an effect on the translation efficiency of any sequence under the control of these mutated Kozak sequences.

Even more advantageously, the invention relates to the abovementioned method, in which said first sequence is a Kozak sequence comprising or consisting of, in its modified version:
when the hybrid nucleic acid molecule is a molecule of deoxyribonucleic acids, especially double-stranded, any one of the following sequences:

| 5′-GGCGCCATGt-3′, | (SEQ ID NO: 38) |
|---|---|
| 5′-GGCACCATGt-3′, | (SEQ ID NO: 39) |
| 5′-GGAGCCATGt-3′, | (SEQ ID NO: 40) |
| 5′-GGAACCATGt-3′, | (SEQ ID NO: 41) |
| 5′-GCCGCCATGt-3′, | (SEQ ID NO: 42) |
| 5′-GCCACCATGt-3′, | (SEQ ID NO: 43) |
| 5′-GCAGCCATGt-3′, | (SEQ ID NO: 44) |
| 5′-GCAACCATGt-3′, | (SEQ ID NO: 45) |
| 5′-CGCGCCATGt-3′, | (SEQ ID NO: 46) |
| 5′-CGCACCATGt-3′, | (SEQ ID NO: 47) |
| 5′-CGAGCCATGt-3′, | (SEQ ID NO: 48) |
| 5′-CGAACCATGt-3′, | (SEQ ID NO: 49) |
| 5′-CCCGCCATGt-3′, | (SEQ ID NO: 50) |
| 5′-CCCACCATGt-3′, | (SEQ ID NO: 51) |
| 5′-CCAGCCATGt-3′, | (SEQ ID NO: 52) |
| 5′-CCAACCATGt-3′, | (SEQ ID NO: 53) |
| 5′-GGCGCCATATGG-3′, | (SEQ ID NO: 54) |
| 5′-GGCACCATATGG-3′, | (SEQ ID NO: 55) |
| 5′-GGAGCCATATGG-3′, | (SEQ ID NO: 56) |
| 5′-GGAACCATATGG-3′, | (SEQ ID NO: 57) |
| 5′-GCCGCCATATGG-3′, | (SEQ ID NO: 58) |
| 5′-GCCACCATATGG-3′, | (SEQ ID NO: 59) |
| 5′-GCAGCCATATGG-3′, | (SEQ ID NO: 60) |
| 5′-GCAACCATATGG-3′, | (SEQ ID NO: 61) |
| 5′-CGCGCCATATGG-3′, | (SEQ ID NO: 62) |
| 5′-CGCACCATATGG-3′, | (SEQ ID NO: 63) |
| 5′-CGAGCCATATGG-3′, | (SEQ ID NO: 64) |
| 5′-CGAACCATATGG-3′, | (SEQ ID NO: 65) |
| 5′-CCCGCCATATGG-3′, | (SEQ ID NO: 66) |
| 5′-CCCACCATATGG-3′, | (SEQ ID NO: 67) |
| 5′-CCAGCCATATGG-3′, | (SEQ ID NO: 68) |
| and | |
| 5/-3;, | SEQ ID NO: 69) |

and
when the hybrid nucleic acid molecule is a molecule of ribonucleic acids, especially single-stranded, any one of the sequences:

| 5′-GGCGCCAUGU-3′, | (SEQ ID NO: 70) |
|---|---|
| 5′-GGCACCAUGU-3′, | (SEQ ID NO: 71) |
| 5′-GGAGCCAUGU-3′, | (SEQ ID NO: 72) |
| 5′-GGAACCAUGU-3′, | (SEQ ID NO: 73) |
| 5′-GCCGCCAUGU-3′, | (SEQ ID NO: 74) |
| 5′-GCCACCAUGU-3′, | (SEQ ID NO: 75) |
| 5′-GCAGCCAUGU-3′, | (SEQ ID NO: 76) |
| 5′-GCAACCAUGU-3′, | (SEQ ID NO: 77) |
| 5′-CGCGCCAUGU-3′, | (SEQ ID NO: 78) |
| 5′-CGCACCAUGU-3′, | (SEQ ID NO: 79) |
| 5′-CGAGCCAUGU-3′, | (SEQ ID NO: 80) |
| 5′-CGAACCAUGU-3′, | (SEQ ID NO: 81) |
| 5′-CCCGCCAUGU-3′, | (SEQ ID NO: 82) |
| 5′-CCCACCAUGU-3′, | (SEQ ID NO: 83) |
| 5′-CCAGCCAUGU-3′, | (SEQ ID NO: 84) |
| 5′-CCAACCAUGU-3′, | (SEQ ID NO: 85) |
| 5′-GGCGCCAUAUGG-3′, | (SEQ ID NO: 86) |
| 5′-GGCACCAUAUGG-3′, | (SEQ ID NO: 87) |
| 5′-GGAGCCAUAUGG-3′, | (SEQ ID NO: 88) |
| 5′-GGAACCAUAUGG-3′, | (SEQ ID NO: 89) |
| 5′-GCCGCCAUAUGG-3′, | (SEQ ID NO: 90) |
| 5′-GCCACCAUAUGG-3′, | (SEQ ID NO: 91) |
| 5′-GCAGCCAUAUGG-3′, | (SEQ ID NO: 92) |
| 5′-GCAACCAUAUGG-3′, | (SEQ ID NO: 93) |
| 5′-CGCGCCAUAUGG-3′, | (SEQ ID NO: 94) |
| 5′-CGCACCAUAUGG-3′, | (SEQ ID NO: 95) |
| 5′-CGAGCCAUAUGG-3′, | (SEQ ID NO: 96) |
| 5′-CGAACCAUAUGG-3′, | (SEQ ID NO: 97) |
| 5′-CCCGCCAUAUGG-3′, | (SEQ ID NO: 98) |
| 5′-CCCACCAUAUGG-3′, | (SEQ ID NO: 99) |
| 5′-CCAGCCAUAUGG-3′, | (SEQ ID NO: 100) |
| and | |
| 5/-3;. | SEQ ID NO: 101) |

Even more advantageously, the invention relates to the abovementioned method, in which said first sequence is a Kozak sequence comprising or consisting of, in its modified version, any one of the sequences SEQ ID NO: 38 to 101.

Still more advantageously, the invention relates to the abovementioned method, in which said first sequence is a Kozak sequence comprising or consisting of, in its modified version, any one of the sequences: SEQ ID NO: 43, SEQ ID NO: 46, SEQ ID NO: 52, SEQ ID NO: 59, SEQ ID NO: 62, SEQ ID NO: 68, SEQ ID NO: 75, SEQ ID NO: 78, SEQ ID NO: 84, SEQ ID NO: 91, SEQ ID NO: 94, and SEQ ID NO: 100.

The invention thus advantageously relates to a method for screening interfering nucleic acids increasing:
gene expression and/or
the activity of genes and/or of ribonucleic acids transcribed from said genes,
said interfering nucleic acids having at least partial sequence complementarity with said gene or said RNA, and
said method comprising a step of introducing, into a eukaryotic cell, especially capable of RNA interference, a hybrid nucleic acid molecule comprising:
a first non-coding sequence intended to initiate translation, said first sequence comprising or consisting of the sequence SEQ ID NO: 1, said first sequence being modified,
a second sequence at least partially complementary to the sequence of said interfering nucleic acids to be screened, and
a third nucleotide sequence encoding at least one determined peptide, said third sequence being under cis translational control of the first sequence,
said first modified sequence comprising or consisting of any one of the sequences SEQ ID NO: 38 to 101, and especially SEQ ID NO: 43, SEQ ID NO: 46, SEQ ID NO: 52, SEQ ID NO: 59, SEQ ID NO: 62, SEQ ID NO: 68, SEQ ID NO: 75, SEQ ID NO: 78, SEQ ID NO: 84, SEQ ID NO: 91, SEQ ID NO: 94, and SEQ ID NO: 100.

The above definitions apply mutatis mutandis.

Thus, the invention advantageously relates to a method for screening interfering nucleic acids increasing:
gene expression and/or
the activity of genes and/or of ribonucleic acids transcribed from said genes,
said interfering nucleic acids having at least partial sequence complementarity with said gene or said RNA, and
said method comprising a step of introducing, into a eukaryotic cell, especially capable of RNA interference, a hybrid nucleic acid molecule comprising:
a first non-coding sequence intended to initiate translation,
a second sequence at least partially complementary to the sequence of said interfering nucleic acids to be screened, and
a third nucleotide sequence encoding at least one determined peptide, said third sequence being under cis translational control of the first sequence,
said first modified sequence comprising or consisting of any one of the sequences SEQ ID NO: 38 to 101, and especially SEQ ID NO: 43, SEQ ID NO: 46, SEQ ID NO: 52, SEQ ID NO: 59, SEQ ID NO: 62, SEQ ID NO: 68, SEQ ID NO: 75, SEQ ID NO: 78, SEQ ID NO: 84, SEQ ID NO: 91, SEQ ID NO: 94, and SEQ ID NO: 100.

The invention even more advantageously relates to the abovementioned method, in which said second sequence comprises from 18 to 10 000 nucleotides at least partially complementary to the sequence of said interfering nucleic acids to be screened, especially from 18 to 1000, in particular from 18 to 500, more particularly from 18 to 100 consecutive nucleotides at least partially complementary to the sequence of said interfering nucleic acids to be screened.

It is desirable, to multiply the screening chances of the interfering nucleic acids according to the invention, to have a hybrid nucleic acid molecule which has a third sequence of large size, which will never be less than 18 nucleotides, which is the minimum size of a small interfering RNA.

An advantageous size for the third sequence is from 18 to 500 nucleotides, which means that the sequence may comprise 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 nucleotides.

Advantageously, the invention relates to a method as defined above, in which said at least one peptide is a natural or recombinant protein which is tagged or untagged, especially an autofluorescent protein.

The third sequence therefore encodes one or more peptides, and especially one or more proteins which may be tagged by means of immunogenic peptides such as the FLAG, HA, V5, Myc or His tags, or tagged with fluorescent proteins such as GFP, CFP, RFP, mCherry, etc. This list is nonlimiting and in no way restricts the scope of the invention.

More advantageously, the peptides used are eGFP encoded by the sequence SEQ ID NO: 102, murine cyclin D1 (CD1) encoded by the sequence SEQ ID NO: 103, murine HRas protein encoded by the sequence SEQ ID NO: 104 or exportin 1 (XPO) encoded by the sequence SEQ ID NO: 105. The advantageous tags are the following: the FLAG tag encoded by the sequence SEQ ID NO: 106, the HA tag encoded by the sequence SEQ ID NO: 107, the Ntag tag encoded by the sequence SEQ ID NO: 108, the V5 tag encoded by the sequence SEQ ID NO: 109, the Myc tag encoded by the sequence SEQ ID NO: 110, or else the Ctag tag encoded by the sequence SEQ ID NO: 111.

Thus, the tagged peptides which may be used within the context of the invention are especially: Myc-XPO encoded by the sequence SEQ ID NO: 112, XPO-V5 encoded by the sequence SEQ ID NO: 113, Myc-XPO-V5 encoded by the sequence SEQ ID NO: 114, Ha-CD1 encoded by the sequence SEQ ID NO: 115.

The invention also relates to a hybrid nucleic acid molecule comprising:
a first non-coding sequence intended to initiate translation,
a second sequence at least partially complementary to at least one interfering nucleic acid, and
a third nucleotide sequence encoding at least one determined peptide, said third sequence being under cis translational control of the first sequence,
said first sequence being modified, by substitution, deletion or addition of at least one nucleotide, such that the level of translation of said at least one peptide is reduced by at least 10% relative to the level of translation of said at least one peptide under control of said first sequence in its unmodified, especially optimal, version.

Such hybrid nucleic acid molecules are novel and do not exist in the natural state because they are artificial molecules consisting of fragments of molecules originating from different genomic origins and loci.

Advantageously, the invention relates to a hybrid nucleic acid molecule as defined above, in which said first sequence is a transcription initiation sequence of Kozak type, downstream of an internal ribosome entry site or IRES, or of a cap (5′cap).

In another advantageous embodiment, the invention relates to an above-mentioned hybrid nucleic acid molecule, in which said first sequence is a Kozak sequence represented, in its unmodified version, by the following sequence:
5′-ssmRccA(T/U)GG-3′ (SEQ ID NO: 1)
   in which R represents a purine, s represents G or C and m represents A/U or C.

Advantageously, the invention relates to an abovementioned hybrid nucleic acid molecule, in which said first sequence is a Kozak sequence comprising or consisting of, in its unmodified version, one of the following sequences: SEQ ID NO: 4 to SEQ ID NO: 35.

In another advantageous embodiment, the invention relates to a hybrid nucleic acid molecule as defined above, in which said first modified sequence is chosen from:
when the hybrid nucleic acid molecule a molecule of deoxyribonucleic acids, especially double-stranded, any one of the sequences SEQ ID NO: 38 to 69, and
when the hybrid nucleic acid molecule a molecule of ribonucleic acids, especially single-stranded, any one of the sequences SEQ ID NO: 70 to 101.

Even more advantageously, the invention relates to a hybrid nucleic acid molecule defined above, said nucleic acid molecule being chosen from molecules of the following sequence: SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127 and SEQ ID NO: 128.

KOZopt-HA-ras-Flag
SEQ ID NO: 116:

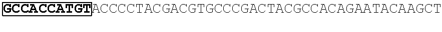

TGTGGTGGTGGGCGCTGGAGGCGTGGGAAAGAGTGCCCTGACCATCCAGC
TGATCCAGAACCACTTTGTGGACGAGTATGATCCCACTATAGAGGACTCC
TACCGGAAACAGGTGGTCATTGATGGGGAGACATGTCTACTGGACATCTT
AGACACAGCAGGTCAAGAAGAGTATAGTGCCATGCGGGACCAGTACATGC
GCACAGGGGAGGGCTTCCTCTGTGTATTTGCCATCAACAACACCAAGTCC
TTCGAGGACATCCATCAGTACAGGGAGCAGATCAAGCGGGTGAAAGATTC
AGATGATGTGCCAATGGTGCTGGTGGGCAACAAGTGTGACCTGGCTGCTC
GCACTGTTGAGTCTCGGCAGGCCCAGGACCTTGCTCGCAGCTATGGCATC
CCCTACATTGAAACATCAGCCAAGACCCGGCAGGGCGTGGAGGATGCCTT
CTATACACTAGTCCGTGAGATTCGGCAGCATAAATTGCGGAAACTGAACC
CACCCGATGAGAGTGGTCCTGGCTGCATGAGCTGCAAATGTGTGCTGTCC
GACTACAAGGACGACGATGACAAG
KOZopt-HA-ras^{G12V}-Flag
SEQ ID NO: 117:

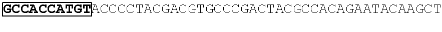

TGTGGTGGTGGGCGCTGtAGGCGTGGGAAAGAGTGCCCTGACCATCCAGC
TGATCCAGAACCACTTTGTGGACGAGTATGATCCCACTATAGAGGACTCC
TACCGGAAACAGGTGGTCATTGATGGGGAGACATGTCTACTGGACATCTT
AGACACAGCAGGTCAAGAAGAGTATAGTGCCATGCGGGACCAGTACATGC
GCACAGGGGAGGGCTTCCTCTGTGTATTTGCCATCAACAACACCAAGTCC
TTCGAGGACATCCATCAGTACAGGGAGCAGATCAAGCGGGTGAAAGATTC
AGATGATGTGCCAATGGTGCTGGTGGGCAACAAGTGTGACCTGGCTGCTC
GCACTGTTGAGTCTCGGCAGGCCCAGGACCTTGCTCGCAGCTATGGCATC
CCCTACATTGAAACATCAGCCAAGACCCGGCAGGGCGTGGAGGATGCCTT
CTATACACTAGTCCGTGAGATTCGGCAGCATAAATTGCGGAAACTGAACC
CACCCGATGAGAGTGGTCCTGGCTGCATGAGCTGCAAATGTGTGCTGTCC
GACTACAAGGACGACGATGACAAG
mKoz-AT-mCD1-Ctag
SEQ ID NO: 118:

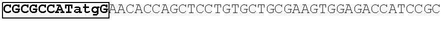

CGCGCGTACCCTGACACCAATCTCCTCAACGACCGGGTGCTGCGAGCCAT
GCTCAAGACGGAGGAGACCTGTGCGCCCTCCGTATCTTACTTCAAGTGCG
TGCAGAAGGAGATTGTGCCATCCATGCGGAAAATCGTGGCCACCTGGATG
CTGGAGGTCTGTGAGGAGCAGAAGTGCGAAGAGGAGGTCTTCCCGCTGGC
CATGAACTACCTGGACCGCTTCCTGTCCCTGGAGCCCTTGAAGAAGAGCC
GCCTGCAGCTGCTGGGGGCCACCTGCATGTTCGTGGCCTCTAAGATGAAG
GAGACCATTCCCTTGACTGCCGAGAAGTTGTGCATCTACACTGACAACTC
TATCCGGCCCGAGGAGCTGCTGCAAATGGAACTGCTTCTGGTGAACAAGC
TCAAGTGGAACCTGGCCGCCATGACTCCCCACGATTTCATCGAACACTTC
CTCTCCAAAATGCCAGAGGCGGATGAGAACAAGCAGACCATCCGCAAGCA
TGCACAGACCTTTGTGGCCCTCTGTGCCACAGATGTGAAGTTCATTTCCA
ACCCACCCTCCATGGTAGCTGCTGGGAGCGTGGTGGCTGCGATGCAAGGC
CTGAACCTGGGCAGCCCCAACAACTTCCTCTCCTGCTACCGCACAACGCA
CTTTCTTTCCAGAGTCATCAAGTGTGACCCGGACTGCCTCCGTGCCTGCC
AGGAACAGATTGAAGCCCTTCTGGAGTCAAGCCTGCGCCAGGCCCAGCAG
AACGTCGACCCCAAGGCCACTGAGGAGGAGGGGGAAGTGGAGGAAGAGGC
TGGTCTGGCCTGCACGCCCACCGACGTGCGAGATGTGGACATCgcggccg
ctggaggagactacaaggacgacgatgacaagtcggccgctggaggatac
ccctacgacgtgcccgactacgcc
mKoz-AT-mCD1
SEQ ID NO: 119:

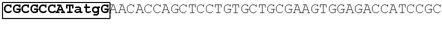

CGCGCGTACCCTGACACCAATCTCCTCAACGACCGGGTGCTGCGAGCCAT
GCTCAAGACGGAGGAGACCTGTGCGCCCTCCGTATCTTACTTCAAGTGCG
TGCAGAAGGAGATTGTGCCATCCATGCGGAAAATCGTGGCCACCTGGATG
CTGGAGGTCTGTGAGGAGCAGAAGTGCGAAGAGGAGGTCTTCCCGCTGGC
CATGAACTACCTGGACCGCTTCCTGTCCCTGGAGCCCTTGAAGAAGAGCC
GCCTGCAGCTGCTGGGGGCCACCTGCATGTTCGTGGCCTCTAAGATGAAG
GAGACCATTCCCTTGACTGCCGAGAAGTTGTGCATCTACACTGACAACTC
TATCCGGCCCGAGGAGCTGCTGCAAATGGAACTGCTTCTGGTGAACAAGC
TCAAGTGGAACCTGGCCGCCATGACTCCCCACGATTTCATCGAACACTTC
CTCTCCAAAATGCCAGAGGCGGATGAGAACAAGCAGACCATCCGCAAGCA
TGCACAGACCTTTGTGGCCCTCTGTGCCACAGATGTGAAGTTCATTTCCA
ACCCACCCTCCATGGTAGCTGCTGGGAGCGTGGTGGCTGCGATGCAAGGC
CTGAACCTGGGCAGCCCCAACAACTTCCTCTCCTGCTACCGCACAACGCA
CTTTCTTTCCAGAGTCATCAAGTGTGACCCGGACTGCCTCCGTGCCTGCC
AGGAACAGATTGAAGCCCTTCTGGAGTCAAGCCTGCGCCAGGCCCAGCAG
AACGTCGACCCCAAGGCCACTGAGGAGGAGGGGGAAGTGGAGGAAGAGGC
TGGTCTGGCCTGCACGCCCACCGACGTGCGAGATGTGGACATC
mKoz-AT-Ntag-mCD1
SEQ ID NO: 120:

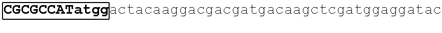

ccctacgacgtgcccgactacgccggaggactcgagGAACACCAGCTCCT
GTGCTGCGAAGTGGAGACCATCCGCCGCGCGTACCCTGACACCAATCTCC
TCAACGACCGGGTGCTGCGAGCCATGCTCAAGACGGAGGAGACCTGTGCG
CCCTCCGTATCTTACTTCAAGTGCGTGCAGAAGGAGATTGTGCCATCCAT
GCGGAAAATCGTGGCCACCTGGATGCTGGAGGTCTGTGAGGAGCAGAAGT
GCGAAGAGGAGGTCTTCCCGCTGGCCATGAACTACCTGGACCGCTTCCTG
TCCCTGGAGCCCTTGAAGAAGAGCCGCCTGCAGCTGCTGGGGGCCACCTG
CATGTTCGTGGCCTCTAAGATGAAGGAGACCATTCCCTTGACTGCCGAGA
AGTTGTGCATCTACACTGACAACTCTATCCGGCCCGAGGAGCTGCTGCAA
ATGGAACTGCTTCTGGTGAACAAGCTCAAGTGGAACCTGGCCGCCATGAC
TCCCCACGATTTCATCGAACACTTCCTCTCCAAAATGCCAGAGGCGGATG
AGAACAAGCAGACCATCCGCAAGCATGCACAGACCTTTGTGGCCCTCTGT
GCCACAGATGTGAAGTTCATTTCCAACCCACCCTCCATGGTAGCTGCTGG
GAGCGTGGTGGCTGCGATGCAAGGCCTGAACCTGGGCAGCCCCAACAACT
TCCTCTCCTGCTACCGCACAACGCACTTTCTTTCCAGAGTCATCAAGTGT
GACCCGGACTGCCTCCGTGCCTGCCAGGAACAGATTGAAGCCCTTCTGGA
GTCAAGCCTGCGCCAGGCCCAGCAGAACGTCGACCCCAAGGCCACTGAGG
AGGAGGGGGAAGTGGAGGAAGAGGCTGGTCTGGCCTGCACGCCCACCGAC
GTGCGAGATGTGGACATC
hKOZ-HA-FLAG-hCD1
SEQ ID NO: 121:

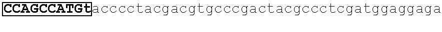

ctacaaggacgacgatgacaagggaggactcgagGAACACCAGCTCCTGT
GCTGCGAAGTGGAAACCATCCGCCGCGCGTACCCCGATGCCAACCTCCTC
AACGACCGGGTGCTGCGGGCCATGCTGAAGGCGGAGGAGACCTGCGCGCC
CTCGGTGTCCTACTTCAAATGTGTGCAGAAGGAGGTCCTGCCGTCCATGC
GGAAGATCGTCGCCACCTGGATGCTGGAGGTCTGCGAGGAACAGAAGTGC
GAGGAGGAGGTCTTCCCGCTGGCCATGAACTACCTGGACCGCTTCCTGTC
GCTGGAGCCCGTGAAAAAGAGCCGCCTGCAGCTGCTGGGGGCCACTTGCA
TGTTCGTGGCCTCTAAGATGAAGGAGACCATCCCCCTGACGGCCGAGAAG
CTGTGCATCTACACCGACAACTCCATCCGGCCCGAGGAGCTGCTGCAAAT
GGAGCTGCTCCTGGTGAACAAGCTCAAGTGGAACCTGGCCGCAATGACCC
CGCACGATTTCATTGAACACTTCCTCTCCAAAATGCCAGAGGCGGAGGAG
AACAAACAGATCATCCGCAAACACGCGCAGACCTTCGTTGCCCTCTGTGC
CACAGATGTGAAGTTCATTTCCAATCCGCCCTCCATGGTGGCAGCGGGGA
GCGTGGTGGCCGCAGTGCAAGGCCTGAACCTGAGGAGCCCCAACAACTTC
CTGTCCTACTACCGCCTCACACGCTTCCTCTCCAGAGTGATCAAGTGTGA
CCCGGACTGCCTCCGGGCCTGCCAGGAGCAGATCGAAGCCCTGCTGGAGT
CAAGCCTGCGCCAGGCCCAGCAGAACATGGACCCCAAGGCCGCCGAGGAG
GAGGAAGAGGAGGAGGAGGAGGTGGACCTGGCTTGCACACCCACCGACGT
GCGGGACGTGGACATC
hKoz-AT-FLAG-hCD1-HA
SEQ ID NO: 122:

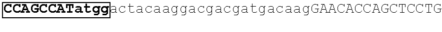

TGCTGCGAAGTGGAAACCATCCGCCGCGCGTACCCCGATGCCAACCTCCT
CAACGACCGGGTGCTGCGGGCCATGCTGAAGGCGGAGGAGACCTGCGCGC
CCTCGGTGTCCTACTTCAAATGTGTGCAGAAGGAGGTCCTGCCGTCCATG
CGGAAGATCGTCGCCACCTGGATGCTGGAGGTCTGCGAGGAACAGAAGTG
CGAGGAGGAGGTCTTCCCGCTGGCCATGAACTACCTGGACCGCTTCCTGT
CGCTGGAGCCCGTGAAAAAGAGCCGCCTGCAGCTGCTGGGGGCCACTTGC
ATGTTCGTGGCCTCTAAGATGAAGGAGACCATCCCCCTGACGGCCGAGAA
GCTGTGCATCTACACCGACAACTCCATCCGGCCCGAGGAGCTGCTGCAAA
TGGAGCTGCTCCTGGTGAACAAGCTCAAGTGGAACCTGGCCGCAATGACC
CCGCACGATTTCATTGAACACTTCCTCTCCAAAATGCCAGAGGCGGAGGA
GAACAAACAGATCATCCGCAAACACGCGCAGACCTTCGTTGCCCTCTGTG
CCACAGATGTGAAGTTCATTTCCAATCCGCCCTCCATGGTGGCAGCGGGG
AGCGTGGTGGCCGCAGTGCAAGGCCTGAACCTGAGGAGCCCCAACAACTT
CCTGTCCTACTACCGCCTCACACGCTTCCTCTCCAGAGTGATCAAGTGTG
ACCCGGACTGCCTCCGGGCCTGCCAGGAGCAGATCGAAGCCCTGCTGGAG
TCAAGCCTGCGCCAGGCCCAGCAGAACATGGACCCCAAGGCCGCCGAGGA
GGAGGAAGAGGAGGAGGAGGAGGTGGACCTGGCTTGCACACCCACCGACG
TGCGGGACGTGGACATCtacccctacgacgtgcccgactacgcc
hKoz-AT-Ntag-hCD1
SEQ ID NO: 123:

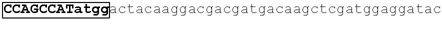

ccctacgacgtgcccgactacgccggaggactcgagGAACACCAGCTCCT
GTGCTGCGAAGTGGAAACCATCCGCCGCGCGTACCCCGATGCCAACCTCC
TCAACGACCGGGTGCTGCGGGCCATGCTGAAGGCGGAGGAGACCTGCGCG
CCCTCGGTGTCCTACTTCAAATGTGTGCAGAAGGAGGTCCTGCCGTCCAT
GCGGAAGATCGTCGCCACCTGGATGCTGGAGGTCTGCGAGGAACAGAAGT
GCGAGGAGGAGGTCTTCCCGCTGGCCATGAACTACCTGGACCGCTTCCTG
TCGCTGGAGCCCGTGAAAAAGAGCCGCCTGCAGCTGCTGGGGGCCACTTG
CATGTTCGTGGCCTCTAAGATGAAGGAGACCATCCCCCTGACGGCCGAGA
AGCTGTGCATCTACACCGACAACTCCATCCGGCCCGAGGAGCTGCTGCAA
ATGGAGCTGCTCCTGGTGAACAAGCTCAAGTGGAACCTGGCCGCAATGAC
CCCGCACGATTTCATTGAACACTTCCTCTCCAAAATGCCAGAGGCGGAGG
AGAACAAACAGATCATCCGCAAACACGCGCAGACCTTCGTTGCCCTCTGT
GCCACAGATGTGAAGTTCATTTCCAATCCGCCCTCCATGGTGGCAGCGGG
GAGCGTGGTGGCCGCAGTGCAAGGCCTGAACCTGAGGAGCCCCAACAACT
TCCTGTCCTACTACCGCCTCACACGCTTCCTCTCCAGAGTGATCAAGTGT
GACCCGGACTGCCTCCGGGCCTGCCAGGAGCAGATCGAAGCCCTGCTGGA
GTCAAGCCTGCGCCAGGCCCAGCAGAACATGGACCCCAAGGCCGCCGAGG
AGGAGGAAGAGGAGGAGGAGGAGGTGGACCTGGCTTGCACACCCACCGAC
GTGCGGGACGTGGACATC
FLAG-KOZopt-AT-mCD1-HA
SEQ ID NO: 124

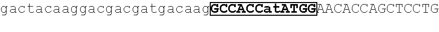

TGCTGCGAAGTGGAGACCATCCGCCGCGCGTACCCTGACACCAATCTCCT
CAACGACCGGGTGCTGCGAGCCATGCTCAAGACGGAGGAGACCTGTGCGC
CCTCCGTATCTTACTTCAAGTGCGTGCAGAAGGAGATTGTGCCATCCATG
CGGAAAATCGTGGCCACCTGGATGCTGGAGGTCTGTGAGGAGCAGAAGTG
CGAAGAGGAGGTCTTCCCGCTGGCCATGAACTACCTGGACCGCTTCCTGT
CCCTGGAGCCCTTGAAGAAGAGCCGCCTGCAGCTGCTGGGGGCCACCTGC
ATGTTCGTGGCCTCTAAGATGAAGGAGACCATTCCCTTGACTGCCGAGAA
GTTGTGCATCTACACTGACAACTCTATCCGGCCCGAGGAGCTGCTGCAAA
TGGAACTGCTTCTGGTGAACAAGCTCAAGTGGAACCTGGCCGCCATGACT
CCCCACGATTTCATCGAACACTTCCTCTCCAAAATGCCAGAGGCGGATGA
GAACAAGCAGACCATCCGCAAGCATGCACAGACCTTTGTGGCCCTCTGTG
CCACAGATGTGAAGTTCATTTCCAACCCACCCTCCATGGTAGCTGCTGGG
AGCGTGGTGGCTGCGATGCAAGGCCTGAACCTGGGCAGCCCCAACAACTT
CCTCTCCTGCTACCGCACAACGCACTTTCTTTCCAGAGTCATCAAGTGTG
ACCCGGACTGCCTCCGTGCCTGCCAGGAACAGATTGAAGCCCTTCTGGAG
TCAAGCCTGCGCCAGGCCCAGCAGAACGTCGACCCCAAGGCCACTGAGGA
GGAGGGGGAAGTGGAGGAAGAGGCTGGTCTGGCCTGCACGCCCACCGACG
TGCGAGATGTGGACATCTACCCCTACGACGTGCCCGACTACGCC
KOZopt-AT-mCD1-HA-STOP-FLAG
SEQ ID NO: 125

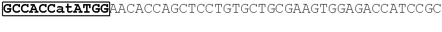

CGCGCGTACCCTGACACCAATCTCCTCAACGACCGGGTGCTGCGAGCCAT
GCTCAAGACGGAGGAGACCTGTGCGCCCTCCGTATCTTACTTCAAGTGCG
TGCAGAAGGAGATTGTGCCATCCATGCGGAAAATCGTGGCCACCTGGATG
CTGGAGGTCTGTGAGGAGCAGAAGTGCGAAGAGGAGGTCTTCCCGCTGGC
CATGAACTACCTGGACCGCTTCCTGTCCCTGGAGCCCTTGAAGAAGAGCC
GCCTGCAGCTGCTGGGGGCCACCTGCATGTTCGTGGCCTCTAAGATGAAG
GAGACCATTCCCTTGACTGCCGAGAAGTTGTGCATCTACACTGACAACTC
TATCCGGCCCGAGGAGCTGCTGCAAATGGAACTGCTTCTGGTGAACAAGC
TCAAGTGGAACCTGGCCGCCATGACTCCCCACGATTTCATCGAACACTTC
CTCTCCAAAATGCCAGAGGCGGATGAGAACAAGCAGACCATCCGCAAGCA
TGCACAGACCTTTGTGGCCCTCTGTGCCACAGATGTGAAGTTCATTTCCA
ACCCACCCTCCATGGTAGCTGCTGGGAGCGTGGTGGCTGCGATGCAAGGC
CTGAACCTGGGCAGCCCCAACAACTTCCTCTCCTGCTACCGCACAACGCA
CTTTCTTTCCAGAGTCATCAAGTGTGACCCGGACTGCCTCCGTGCCTGCC
AGGAACAGATTGAAGCCCTTCTGGAGTCAAGCCTGCGCCAGGCCCAGCAG
AACGTCGACCCCAAGGCCACTGAGGAGGAGGGGGAAGTGGAGGAAGAGGC
TGGTCTGGCCTGCACGCCCACCGACGTGCGAGATGTGGACATCTACCCAC
GACGTGCCCGACTACGCCTGAgactacaaggacgacgatgacaag
FLAG-hKoz-AT-hCD1-STOP-HA
SEQ ID NO: 126

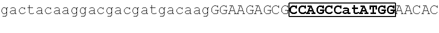

CAGCTCCTGTGCTGCGAAGTGGAAACCATCCGCCGCGCGTACCCCGATGC
CAACCTCCTCAACGACCGGGTGCTGCGGGCCATGCTGAAGGCGGAGGAGA
CCTGCGCGCCCTCGGTGTCCTACTTCAAATGTGTGCAGAAGGAGGTCCTG
CCGTCCATGCGGAAGATCGTCGCCACCTGGATGCTGGAGGTCTGCGAGGA
ACAGAAGTGCGAGGAGGAGGTCTTCCCGCTGGCCATGAACTACCTGGACC
GCTTCCTGTCGCTGGAGCCCGTGAAAAAGAGCCGCCTGCAGCTGCTGGGG
GCCACTTGCATGTTCGTGGCCTCTAAGATGAAGGAGACCATCCCCCTGAC
GGCCGAGAAGCTGTGCATCTACACCGACAACTCCATCCGGCCCGAGGAGC
TGCTGCAAATGGAGCTGCTCCTGGTGAACAAGCTCAAGTGGAACCTGGCC
GCAATGACCCCGCACGATTTCATTGAACACTTCCTCTCCAAAATGCCAGA
GGCGGAGGAGAACAAACAGATCATCCGCAAACACGCGCAGACCTTCGTTG
CCCTCTGTGCCACAGATGTGAAGTTCATTTCCAATCCGCCCTCCATGGTG
GCAGCGGGGAGCGTGGTGGCCGCAGTGCAAGGCCTGAACCTGAGGAGCCC
CAACAACTTCCTGTCCTACTACCGCCTCACACGCTTCCTCTCCAGAGTGA
TCAAGTGTGACCCGGACTGCCTCCGGGCCTGCCAGGAGCAGATCGAAGCC
CTGCTGGAGTCAAGCCTGCGCCAGGCCCAGCAGAACATGGACCCCAAGGC
CGCCGAGGAGGAGGAAGAGGAGGAGGAGGAGGTGGACCTGGCTTGCACAC
CCACCGACGTGCGGGACGTGGACATCTGAtacccctacgacgtgcccgac
tacgcc
KOZopt-AT-Myc-CDK4-V5
SEQ ID NO: 127

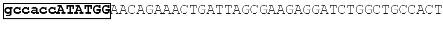

CGATATGAACCCGTGGCTGAAATTGGTGTCGGTGCCTATGGGACGGTGTA
CAAAGCCCGAGATCCCCACAGTGGCCACTTTGTGGCCCTCAAGAGTGTGA
GAGTTCCTAATGGAGGAGCAGCTGGAGGGGGCCTTCCCGTCAGCACAGTT
CGTGAGGTGGCCTTGTTAAGGAGGCTGGAGGCCTTTGAACATCCCAATGT
TGTACGGCTGATGGATGTCTGTGCTACTTCCCGAACTGATCGGGACATCA
AGGTCACCCTAGTGTTTGAGCATATAGACCAGGACCTGAGGACATACCTG
GACAAAGCACCTCCACCGGGCCTGCCGGTTGAGACCATTAAGGATCTAAT
GCGTCAGTTTCTAAGCGGCCTGGATTTTCTTCATGCAAACTGCATTGTTC
ACCGGGACCTGAAGCCAGAGAACATTCTAGTGACAAGTAATGGGACCGTC
AAGCTGGCTGACTTTGGCCTAGCTAGAATCTACAGCTACCAGATGGCCCT
CACGCCTGTGGTGGTTACGCTCTGGTACCGAGCTCCTGAAGTTCTTCTGC
AGTCTACATACGCAACACCCGTGGACATGTGGAGCGTTGGCTGTATCTTT
GCAGAGATGTTCCGTCGGAAGCCTCTCTTCTGTGGAAACTCTGAAGCCGA
CCAGTTGGGGAAAATCTTTGATCTCATTGGATTGCCTCCAGAAGACGACT
GGCCTCGAGAGGTATCTCTACCTCGAGGAGCCTTTGCCCCCAGAGGGCCT
CGGCCAGTGCAGTCAGTGGTGCCAGAGATGGAGGAGTCTGGAGCGCAGCT
GCTACTGGAAATGCTGACCTTTAACCCACATAAGCGAATCTCTGCCTTCC
GAGCCCTGCAGCACTCCTACCTGCACAAGGAGGAAAGCGACGCAGAGGGC
AAACCGATTCCGAACCCGCTGCTGGGCCTGGATAGCACC
FLAG-KOZopt-AT-Gly-Ras-HA
SEQ ID NO: 128

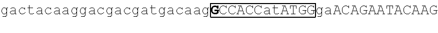

CTTGTGGTGGTGGGCGCTGGAGGCGTGGGAAAGAGTGCCCTGACCATCCA
GCTGATCCAGAACCACTTTGTGGACGAGTATGATCCCACTATAGAGGACT
CCTACCGGAAACAGGTGGTCATTGATGGGGAGACATGTCTACTGGACATC
TTAGACACAGCAGGTCAAGAAGAGTATAGTGCCATGCGGGACCAGTACAT
GCGCACAGGGGAGGGCTTCCTCTGTGTATTTGCCATCAACAACACCAAGT
CCTTCGAGGACATCCATCAGTACAGGGAGCAGATCAAGCGGGTGAAAGAT
TCAGATGATGTGCCAATGGTGCTGGTGGGCAACAAGTGTGACCTGGCTGC
TCGCACTGTTGAGTCTCGGCAGGCCCAGGACCTTGCTCGCAGCTATGGCA
TCCCCTACATTGAAACATCAGCCAAGACCCGGCAGGGCGTGGAGGATGCC
TTCTATACACTAGTCCGTGAGATTCGGCAGCATAAATTGCGGAAACTGAA
CCCACCCGATGAGAGTGGTCCTGGCTGCATGAGCTGCAAATGTGTGCTGT
CCTACCCCTACGACGTGCCCGACTACGCC

In the above table, the first sequence, in its mutated version, is indicated by a box.

These examples of hybrid nucleic acid molecules illustrate nonlimitingly the different possibilities covered by the invention, and for example:
in the sequence SEQ ID NO: 128, the first sequence is contained within a box, the second sequence is in 3′ of the first sequence, and the third sequence is in 5′ of the first sequence. This hybrid nucleic acid molecule ideally makes it possible to select nucleic acids increasing the expression of the FLAG peptide.
in the sequence SEQ ID NO: 126 or 125, the first sequence is contained within a box, the second sequence is in 3′ of the first sequence, and the third sequence is in 3′ of the second sequence. This hybrid nucleic acid molecule ideally makes it possible to select nucleic acids increasing the expression of the HA or FLAG peptide, respectively.

Of course, as mentioned above, the second and the third sequence may be superimposed, that is to say that a portion of the second sequence corresponds to the third sequence. Thus, the hybrid nucleic acid molecules as illustrated by the sequences SEQ ID NO: 116 to 128 also make it possible to select interfering nucleic acids against the murine or human cyclin D1 protein, or else the Ras protein.

Each of the above-mentioned sequences, when no STOP is mentioned (codon underlined in the sequences SEQ ID NO: 125 and 126) in its name (under the sequence number) is terminated by a stop codon TAG, TAA or TGA.

Due to base complementarity, those skilled in the art are able to determine the RNAs corresponding to the above sequences.

Advantageously, the above-mentioned hybrid nucleic acid molecule is contained in a vector, especially a eukaryotic vector.

More advantageously, the vectors essentially consist of the following sequences: pBABE, especially represented by one of the sequences SEQ ID NO: 129 or 130, or MSCV, especially represented by the sequence SEQ ID NO: 131.

In addition, the invention relates to a eukaryotic cell comprising at least one hybrid nucleic acid molecule as defined above. The invention also relates to an animal, especially a mammal, in particular a rodent, comprising at least one hybrid nucleic acid molecule as defined above.

The invention encompasses any type of eukaryotic cell capable of RNA interference. Those skilled in the art, with their general knowledge of eukaryotic cells cultured in vitro, are capable of readily identifying the appropriate cells and of determining the methods for transformation or transfection in order to introduce the nucleic acid molecule defined above therein.

The invention moreover relates to an intermediate hybrid nucleic acid molecule comprising:
a first non-coding sequence intended to initiate translation, as defined above,
a third nucleotide sequence encoding at least one determined peptide, said third sequence being under cis translational control of the first sequence, as defined above,
and at least one site for cleavage by a restriction enzyme, enabling the insertion of a nucleic acid molecule having a sequence complementary to an interfering nucleic acid,
said first sequence being modified, by substitution, deletion or addition of at least one nucleotide, such that the level of translation of said at least one peptide is reduced by at least 10% relative to the level of translation of said at least one peptide under control of said first sequence in its unmodified, especially optimal, version.

Advantageously, the invention relates to the above-mentioned method, in which said first sequence is a Kozak sequence represented, in its unmodified version, by the following sequence:

| | |
|---|---|
| 5′-ssmRccA(T/U)GG-3′ | (SEQ ID NO: 1) |

in which R represents a purine, s represents G or C and m represents A/U or C, and especially in which said first sequence is a Kozak sequence comprising or consisting of, in its modified version, one of the following sequences: SEQ ID NO: 4 or SEQ ID NO: 5.

This intermediate hybrid nucleic acid molecule is in fact the base structure of the above-mentioned hybrid nucleic acid molecule, said at least one site for cleavage by a restriction enzyme enabling the cloning of said second sequence according to the gene for which it is desirable to screen interfering nucleic acids increasing the expression of said gene and/or the activity of said gene and/or ribonucleic acids transcribed from said gene.

The invention also relates to the use of at least one nucleic acid molecule as defined above, for screening, especially in vitro, interfering nucleic acids increasing gene expression and/or the activity of genes and/or of ribonucleic acids transcribed from said genes.

The invention moreover relates to a kit, or a case, comprising:
at least one hybrid nucleic acid molecule as described above, and
at least one eukaryotic cell.

A case or a kit according to the invention may also comprise:
at least one hybrid nucleic acid molecule as described above, and
means for transforming a eukaryotic cell by said hybrid nucleic acid molecule.

A case or a kit according to the invention may also comprise:
at least one hybrid nucleic acid molecule as described above, and
   means for transforming a eukaryotic cell by said hybrid nucleic acid molecule, and/or
   at least one eukaryotic cell capable of RNA interference.

The transformation means used may be means for transforming eukaryotic cells such as calcium phosphate cell transformation means, means for transformation with liposomes, means for transformation with polycationic agents or else means for transformation by electrolocation or nucleofection.

The invention also relates to a kit, or a case, comprising:
at least one hydride nucleic acid molecule as defined above, said molecule being contained within a eukaryotic cell, and
means for transforming said cell by interfering nucleic acids.

It will be noted that throughout the preceding text, the term cell “transformation” is used in the sense of introducing exogenous nucleic acid molecule(s) into the eukaryotic cell in question. Those skilled in the art will thus understand that this idea of transformation corresponds to the idea of “transfection” commonly used in the art.

The invention will be better understood in light of the figures and examples described below.

### BRIEF DESCRIPTION OF THE DRAWINGS

FIG. 1 schematically describes the different types of hybrid nucleic acid molecules described in the invention. 1 schematically represents the first sequence, 2 schematically represents the second sequence, and 3 schematically represents the third sequence. 3* represents the third sequence into which the second sequence has been inserted. n represents a sequence which is neither the first, nor the second, nor the third sequence.

FIG. 2 represents the diagrams resulting from sequencing of the first sequence of the hybrid nucleic acid molecule having a first, unmutated, sequence (top diagram) and of the first sequence of the hybrid nucleic acid molecule having a first sequence mutated by an insertion of an AT dinucleotide, indicated by the ellipse (top diagram).

FIG. 3 represents the alignment of the hybrid nucleic acid molecule SEQ ID NO: 120 with the sequences of interfering nucleic acid molecules tested. The sequence numbers (SEQ ID) are indicated.

FIG. 4 represents a Western blot produced from cells having the hybrid nucleic acid molecule SEQ ID NO: 120 and transfected with siRNAs SEQ ID NO: 138 (1), SEQ ID NO: 140 (3), SEQ ID NO: 142 (5), SEQ ID NO: 144 (7), control SEQ ID NO: 161/162 (T.), SEQ ID NO: 150 (F3), SEQ ID NO: 152 (F5), SEQ ID NO: 153 (F6) or SEQ ID NO: 154 (F-M). The proteins are revealed with an anti-HA antibody (B.). As control, the protein loading is revealed with an anti-actin antibody (A.).

FIG. 5 represents a Western blot produced from cells having the hybrid nucleic acid molecule SEQ ID NO: 120, non-transfected (−) or transfected with the control siRNAs SEQ ID NO: 161/162 (T.), SEQ ID NO: 146 (F-N), SEQ ID NO: 149 (F2), SEQ ID NO: 148 (F), SEQ ID NO: 142 (5) or SEQ ID NO: 145 (CT). The proteins are revealed with an anti-HA antibody (B.). As control, the protein loading is revealed with an anti-actin antibody (A.).

FIGS. 6A and 6B represent the comparison of the effect of the mutation of the first sequence of the hybrid nucleic acid molecule.

FIG. 6A represents a Western blot produced from cells having the hybrid nucleic acid molecule SEQ ID NO: 136 transfected with the control siRNAs SEQ ID NO: 161/162 (T.), SEQ ID NO: 150 (F3) or SEQ ID NO: 154 (F-M). The proteins are revealed with an anti-HA antibody (B.). As control, the protein loading is revealed with an anti-actin antibody (A.).

FIG. 6B represents a Western blot produced from cells having the hybrid nucleic acid molecule SEQ ID NO: 120 transfected with the control siRNAs SEQ ID NO: 161/162 (T.), SEQ ID NO: 150 (F3) or SEQ ID NO: 154 (F-M). The proteins are revealed with an anti-HA antibody (B.). As control, the protein loading is revealed with an anti-actin antibody (A.).

FIG. 7 represents a histogram of FRET results representing the amount of expression of CD1 in cells having the sequence SEQ ID NO: 1 transfected with one of the following siRNAs: SEQ ID NO: 146 (B), SEQ ID NO: 147 (C), SEQ ID NO: 148 (D), SEQ ID NO: 149 (E), SEQ ID NO: 150 (F), SEQ ID NO: 151 (G), SEQ ID NO: 152 (H), SEQ ID NO: 153 (I), SEQ ID NO: 155 (J), SEQ ID NO: 156 (K), SEQ ID NO: 154 (L), SEQ ID NO: 137 (M), SEQ ID NO: 138 (N), SEQ ID NO: 139 (0), SEQ ID NO: 140 (P), SEQ ID NO: 141 (Q), SEQ ID NO: 142 (R), SEQ ID NO: 143 (S), SEQ ID NO: 144 (T) or SEQ ID NO: 145 (U), compared to cells having the sequence SEQ ID NO: 120 and transfected with the control siRNAs (SEQ ID NO: 161/162). The results are expressed as percentages. As control, the results obtained for non-transfected cells (U) are presented. siRNAs increasing expression are indicated by an arrow.

FIG. 8 represents a Western blot produced from cells having the hybrid nucleic acid molecule SEQ ID NO: 121 and transfected with siRNAs SEQ ID NO: 139 (2), SEQ ID NO: 140 (3), SEQ ID NO: 142 (5), SEQ ID NO: 144 (7), control SEQ ID NO: 161/162 (T.), SEQ ID NO: 150 (F3), SEQ ID NO: 152 (F5), SEQ ID NO: 153 (F6) or SEQ ID NO: 154 (F-M). The proteins are revealed with an anti-HA antibody (B.). As control, the protein loading is revealed with an anti-actin antibody (A.).

FIG. 9 represents a Western blot produced from cells having the hybrid nucleic acid molecule SEQ ID NO: 121 and transfected with siRNAs SEQ ID NO: 139 (2), SEQ ID NO: 140 (3), SEQ ID NO: 142 (5), SEQ ID NO: 144 (7), control SEQ ID NO: 161/162 (T.), SEQ ID NO: 150 (F3), SEQ ID NO: 152 (F5), SEQ ID NO: 153 (F6) or SEQ ID NO: 154 (F-M). The proteins are revealed with an anti-HA antibody (B.). As control, the protein loading is revealed with an anti-actin antibody (A.).

FIG. 10 represents a histogram of FRET results representing the amount of expression of CD1 (in arbitrary units) in cells having a construct with the murine Kozak sequence (A), the murine Kozak sequence mutated by AT insertion (B) or the optimized Kozak sequence (C). The error bars indicate the standard deviation obtained for three independent experiments.

FIG. 11 represents a Western blot produced from cells having a construct with the murine Kozak sequence (A), the murine Kozak sequence mutated by AT insertion (B) or the optimized Kozak sequence (C). The level of cyclin D1 is revealed with an anti-cyclin D1 antibody (1. RB-010-PABX (AB3), Fisher Scientific). As control, the protein loading is revealed with an anti-actin antibody (2. ab6276, Abcam).

FIG. 12 represents a histogram showing the abundance of cyclin D1 messenger RNAs in cells having a construct with the murine Kozak sequence (A), the murine Kozak sequence mutated by AT insertion (B) or the optimized Kozak sequence (C).

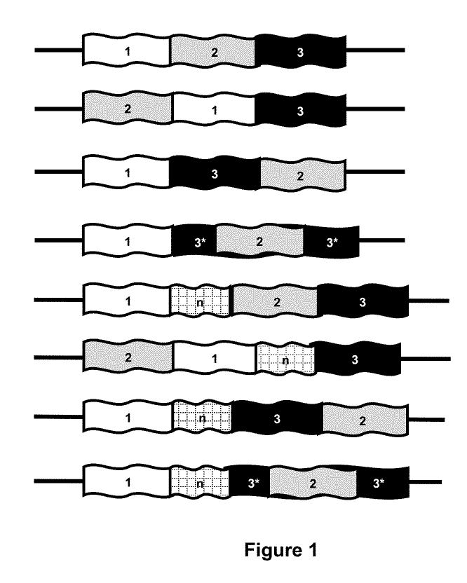

FIG. 13 represents a histogram of FRET results representing the amount of expression of CD1 (in arbitrary units) in cells having one of the following constructs:
N-terminal-tagged cyclin D1 under the control of the murine cyclin D1 Kozak having an AT insertion (Ntag-mKozAT); black bars,
N-terminal-tagged cyclin D1 under the control of the murine cyclin D1 Kozak (Ntag-mKoz); white bars,
C-terminal-tagged cyclin D1 under the control of the murine cyclin D1 AT Kozak (Ctag-mKozAT); gray bars,
C-terminal-tagged cyclin D1 under the control of the murine cyclin D1 Kozak having an AT insertion (Ctag-mKozAT); diagonally striped bars, and
N-terminal-tagged cyclin D1 under the control of the murine cyclin D1 Kozak optimized to increase expression (Ntag-KozOPT); horizontally striped bars,
treated with different siRNAs: irrelevant control (A); siRNA SEQ ID NO: 149/150 (B), siRNA SEQ ID NO: 155 (C), siRNA SEQ ID NO: 154 (D) and siRNA SEQ ID NO: 142 (C).

### DETAILED DESCRIPTION OF THE INVENTION

### EXAMPLES

### Example 1

### Example of a Construction of a Hybrid Nucleic Acid Molecule Comprising the First Sequence SEQ ID NO: 37 (AT Insertion).

In order to obtain a construct comprising a mutated first sequence represented by the sequence SEQ ID NO: 36, the inventors used a strategy of site-directed mutagenesis by introducing, into the Kozak sequence SEQ ID NO: 9, an AT dinucleotide by PCR using the GeneArt® (Life Technology) kit, following the manufacturer's instructions.

Put briefly, the insertion is carried out by means of the template vector comprising the sequence SEQ ID NO: 9 and sense and antisense oligonucleotides containing the AT mutation/insertion;

sense:
(SEQ ID NO: 132)
5′-TGGTACGGCgccaccATatggactacaaggac-3′,
antisense:
(SEQ ID NO: 133)
5′-gtccttgtagtccatATggtggcGCCGTACCA-3′.

The polymerase chain reaction (PCR) is carried out under the following conditions:
Step 1—methylation of the template plasmid: 20 minutes at 37° C.
Step 2—PCR/mutagenesis
a—1 cycle of 2 minutes at 95° C.
b—1 cycle of 30 seconds at 95° C.
   1 cycle of 30 seconds at 60° C.
   1 cycle of 4 minutes at 68° C.
c—Return to b 35 times
d—1 cycle of 5 minutes at 68° C.
e—1 cycle of undefined duration at 4° C. to preserve the reaction product.

The PCR products obtained in this way are then sequenced and the vectors comprising the sequence SEQ ID NO: 59 are selected.

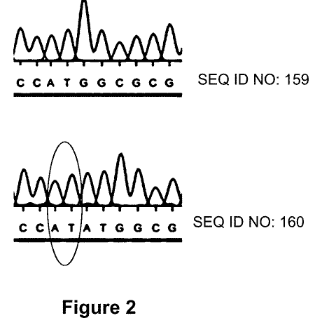

FIG. 2 shows the results of the sequencing.

### Example 2

### Example of Construction of a Hybrid Nucleic Acid Molecule Comprising the First Sequence SEQ ID NO: 36 (G->T Substitution).

In order to obtain a hybrid nucleic acid molecule comprising a first sequence mutated by substitution of the G located just after the ATG by a T, it is sufficient:
1—either to replace the tag by supplying a tag which starts with T instead of G (HA instead of FLAG, for example),
2—or to insert a triplet (1 codon) in order to add an amino acid encoded by a codon which starts with a T. It is necessary to add 3 bases (or a multiple of 3) to retain the open reading frame for translation of the ensuing reporter peptide.

In the case in which the modification of the coding sequence is unimportant, it is also possible to carry out site-directed mutagenesis as indicated in example 1, using the following oligonucleotides:

sense:
(SEQ ID NO: 157)
5′-TGGTACGGCgccaccatgTTRactacaaggac-3′,
R being a purine
antisense:
(SEQ ID NO: 158)
5′-gtccttgtagtYAAcatggtggcGCCGTACCA-3′,
Y being a pyrimidine.

### Example 3

### Example of Transformation of Eukaryotic Cells by the Hybrid Nucleic Acid Molecule

Depending on the experiments to be carried out, several transfection techniques may be used:

a) Transfection with Lipofectamine® 3000 (Invitrogen).

This method makes it possible to rapidly and transiently transfect the cells with the hybrid nucleic acid constructs. The transfection is carried out according to the manufacturer's instructions.

b) Viral infection after production of virus containing the constructs of interest.

In order to obtain cells which stably express a hybrid nucleic acid molecule according to the invention, the inventors made use of viral infection.

The protocol used is as follows:

Day 1: 3×10⁶ 293T cells in exponential growth are seeded in 100 mm dishes with 10 ml of complete medium (DMEM, 10% fetal calf serum, penicillin, streptomycin and L-glutamine) and incubated at 37° C. overnight.

Day 2: 2 to 3 hours before transfection, the culture medium is changed in order to limit pH variations.

The following plasmids are added, in this order, into a tube:
12 μg of MSCV vector or of retroviral vector comprising the hybrid nucleic acid molecule,
6 μg of Gag-pol plasmid, and
2 μg of Eco plasmid (specific viral recognition protein of murine cells, used for safety reasons linked to GMO manipulations).

500 μl of sterile water are then added to the vectors. 500 μl of 2× HBS buffer are then added, and everything is agitated without however being subjected to vortex agitation. Finally, 50 μl of a solution of CaCl₂, pH 5.5, is added, and the mixture is agitated without however being subjected to vortex agitation. The HSB 2× medium is prepared in the following way: 0.8 g of NaCl, 0.027 g of Na₂HPO₄.2H₂O, and 1.2 g of HEPES are dissolved in a volume of 90 ml of distilled water. The pH is adjusted to 7.05 with 0.5 N NaOH, and the volume is adjusted to 100 ml with distilled water. The solution is sterilized by filtering it through a filter with 0.22 μm pores, and the solution is aliquoted by 5 ml before freezing at −20° C. for a maximum duration of one year.

The mixture is left at room temperature for 20 to 30 min with occasional gentle agitation.

The mixture is then added to the culture medium and the cells are incubated at 37° C. overnight.

Day 3: On the morning of the third day, approximately half of the medium is changed. The medium is then conserved at 4° C. The operation is repeated every 6 hours and the supernatant conserved at 4° C. In the evening, the supernatants are mixed and optionally centrifuged at 10 000 rpm overnight.

Day 4: The viruses are recovered, and the medium is changed three times during the day to keep the viruses infectious.

Day 5: The viruses are filtered on a 0.45 μm filter and used to infect NIH3T3 cells for 1 to 2 hours in a volume of 1.5 to 2 ml comprising 8 μg/ml of polybrene. 10 ml of medium are then added and the cells incubated overnight.

Day 6: the medium is changed with 10 ml of fresh medium.

Days 8 and 9: the cells are then analyzed by flow cytometry to test the expression of fluorescent proteins.

The cells are then infected with the molecule.

### Example 4

### Example of Screening of Interfering Nucleic Acid Molecules Increasing Gene Expression and/or the Activity of Genes and/or of Ribonucleic Acids Transcribed from Said Genes

1. Protocol
Cell Culture:
the stable cells expressing the hybrid nucleic acid construct are kept in sub-confluent culture at 37° C., 5% CO₂ in an incubator.
Plating:

On the morning of the transfection of the siRNAs to be screened, the stock cells expressing the hybrid construct are treated with trypsin to detach them from the culture support and seeded in 24-well plates in order to achieve 40 to 80% confluence of adherent cells by the evening.
Preparation of the siRNAs and Transfection:

The siRNAs to be tested are prepared according to the Lipofectamine® RNAiMAX Reagent (Life Technologies) protocol. Put briefly, 1.5 μl of Lipofectamine® are diluted in 25 μl of OPTI-MEM® medium. In parallel, 5 pmol of siRNA in 0.5 μl of sterile water are diluted in 25 μl of OPTI-MEM® medium. The two solutions of OPTI-MEM® are then mixed and incubated for 5 minutes at room temperature.

The preceding 50 μl mixture is then added into each well. The cells are incubated at 37° C. until the following day.
Analysis of the Results:

The following day, the cells are washed with PBS then lyzed by means of a lysis buffer (10 mM TRIS pH=8, 1 mM EDTA, 0.05% NP-40, +/−ROCHE-cOmplete-protease inhibitors) and a step of sonication at a rate of 5 sonication cycles with a bioruptor (Diagenode), composed of 15′ of active sonication (maximum power of the apparatus) followed by 15′ pause, in order to recover the intracellular proteins, especially the fusion protein produced by the hybrid sequence. The protein lysates of the different conditions tested are then standardized by means of DNA or protein quantification, in order to compare an equivalent total amount of material originating from the different treatment conditions. The standardized lysates are then analyzed by Western blot by means of an antibody specific to the translation product of the hybrid construct, or by FRET measurement, or by fluorescence measurement if the translation product of the hybrid construct enables it.

2—Results

a) Mutation by AT Insertion

In a first series of experiments, the inventors carried out screening of interfering molecules according to the invention using the hybrid nucleic acid molecule SEQ ID NO: 220. In this molecule:

| the first sequence is | |
|---|---|
| CGCGCCATatgg, | (SEQ ID NO: 62) |

the second sequence is:

(SEQ ID NO: 134)
ACTACAAGGACGACGATGACAAGCTCGATGGAGGATACCCCTACGACGTG
CCCGACTACGCCGGAGGACTCGAGG,
and corresponds to a FLAG-spacer-HA-spacer
sequence,

and the third sequence is:

(SEQ ID NO: 135)
AACACCAGCTCCTGTGCTGCGAAGTGGAGACCATCCGCCGCGCGTACCCT
GACACCAATCTCCTCAACGACCGGGTGCTGCGAGCCATGCTCAAGACGGA
GGAGACCTGTGCGCCCTCCGTATCTTACTTCAAGTGCGTGCAGAAGGAGA
TTGTGCCATCCATGCGGAAAATCGTGGCCACCTGGATGCTGGAGGTCTGT
GAGGAGCAGAAGTGCGAAGAGGAGGTCTTCCCGCTGGCCATGAACTACCT
GGACCGCTTCCTGTCCCTGGAGCCCTTGAAGAAGAGCCGCCTGCAGCTGC
TGGGGGCCACCTGCATGTTCGTGGCCTCTAAGATGAAGGAGACCATTCCC
TTGACTGCCGAGAAGTTGTGCATCTACACTGACAACTCTATCCGGCCCGA
GGAGCTGCTGCAAATGGAACTGCTTCTGGTGAACAAGCTCAAGTGGAACC
TGGCCGCCATGACTCCCCACGATTTCATCGAACACTTCCTCTCCAAAATG
CCAGAGGCGGATGAGAACAAGCAGACCATCCGCAAGCATGCACAGACCTT
TGTGGCCCTCTGTGCCACAGATGTGAAGTTCATTTCCAACCCACCCTCCA
TGGTAGCTGCTGGGAGCGTGGTGGCTGCGATGCAAGGCCTGAACCTGGGC
AGCCCCAACAACTTCCTCTCCTGCTACCGCACAACGCACTTTCTTTCCAG
AGTCATCAAGTGTGACCCGGACTGCCTCCGTGCCTGCCAGGAACAGATTG
AAGCCCTTCTGGAGTCAAGCCTGCGCCAGGCCCAGCAGAACGTCGACCCC
AAGGCCACTGAGGAGGAGGGGGAAGTGGAGGAAGAGGCTGGTCTGGCCTG
CACGCCCACCGACGTGCGAGATGTGGACATC.

Under the experimental conditions as described above, the inventors tested the following different interfering nucleic acids (siRNAs):

| HA linker Nter: | |
|---|---|
| GAGCUACCUCCUAUGGGGAUG, | (SEQ ID NO: 137) |
| HA: | |
| AUGCUGCACGGGCUGAUGCGG, | (SEQ ID NO: 138) |
| HA 2: | |
| GGGAUGCUGCACGGGCUGAUG, | (SEQ ID NO: 139) |
| HA 3: | |
| AUGGGGAUGCUGCACGGGCUG, | (SEQ ID NO: 140) |
| HA 4: | |
| UGGGGAUGCUGCACGGGCUGA, | (SEQ ID NO: 141) |
| HA 5: | |
| GGGGAUGCUGCACGGGCUGAU, | (SEQ ID NO: 142) |
| HA 6: | |
| GGAUGCUGCACGGGCUGAUGC, | (SEQ ID NO: 143) |
| HA 7: | |
| GAUGCUGCACGGGCUGAUGCG, | (SEQ ID NO: 144) |
| HA Linker Cter: | |
| AGCCGGCGACCUCCUAUGGGG, | (SEQ ID NO: 145) |
| FLAG N: | |
| CUGCUGCUACUGUUCGAGCUA, | (SEQ ID NO: 146) |
| FLAG N2: | |
| UUCCUGCUGCUACUGUUCGAG, | (SEQ ID NO: 147) |
| FLAG: | |
| AUGUUCCUGCUGCUACUGUUC, | (SEQ ID NO: 148) |
| FLAG V2: | |
| CUGAUGUUCCUGCUGCUACUG, | (SEQ ID NO: 149) |
| FLAG 3: | |
| CUGAUGUUCCUGCUGCUACUG, | (SEQ ID NO: 150) |
| FLAG 4: | |
| UGAUGUUCCUGCUGCUACUGU, | (SEQ ID NO: 151) |
| FLAG 5: | |
| GAUGUUCCUGCUGCUACUGUU, | (SEQ ID NO: 152) |
| FLAG 6: | |
| ACCUGAUGUUCCUGCUGCUAC, | (SEQ ID NO: 153) |
| FLAG M: | |
| AUGUUCCUGCUGCUGCUAUUC, | (SEQ ID NO: 154) |
| FLAG + linker Cter: | |
| CUGCUGCUACUGUUCAGCCGG, | (SEQ ID NO: 155) |
| and | |
| FLAG Cter2 ha ct: | |
| UUCCUGCUGCUACUGUUCAGC. | (SEQ ID NO: 156) |

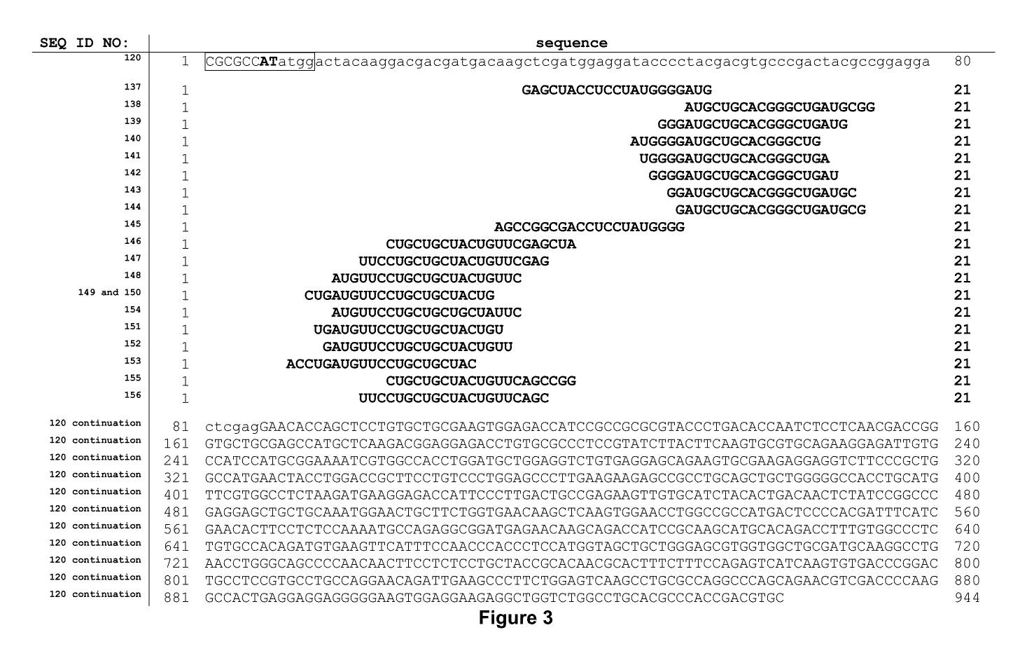

In order to facilitate reading, FIG. 3 represents the alignment of the different interfering nucleic acids (siRNAs) tested are on the sequence of the molecule SEQ ID NO: 120.

As siRNA transfection control, the negative control siRNAs (“scramble”; T.) are also transfected. These siRNAs have the following sense sequence: 5′-UUCUCCGAACGUGUCACGUtt-3′ (SEQ ID NO: 161) and the complementary strand has the following sequence: 5′-ACGUGACACAUUCGGAGAAtt-3′ (SEQ ID NO: 162).

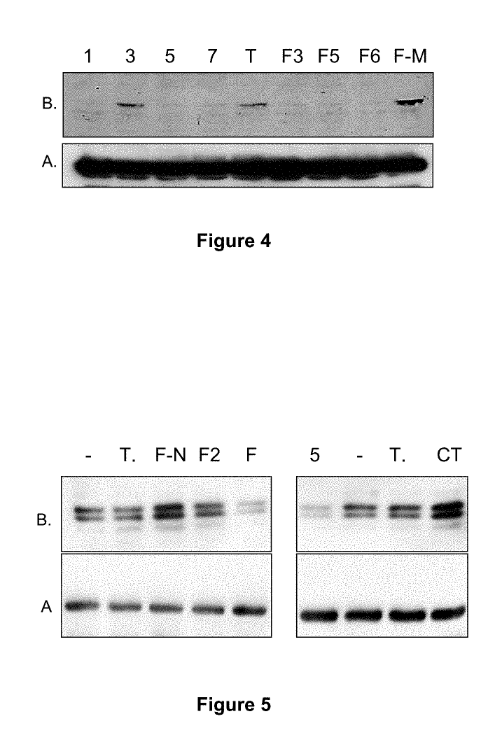

The results obtained by Western blot are represented in FIG. 4.

From this figure it is observed that from the different siRNAs tested, the siRNA FLAG-M (SEQ ID NO: 154) makes it possible to detect a higher level of expression of the marker peptide CD1 than the level observed with an irrelevant control.

In order to confirm that the position of the third sequence did not have any effect on the screening of interfering nucleic acids increasing expression, the inventors used the hybrid nucleic acid molecule SEQ ID NO: 118.

The results obtained by Western blot are represented in FIG. 5.

From this figure it can be seen that from the different siRNAs tested, the siRNA FLAG-N (SEQ ID NO: 146) and the siRNA HA-CT (SEQ ID NO: 145) make it possible to detect a higher level of expression of the marker peptide CD1 than the level observed with an irrelevant control.

Finally, the inventors confirmed that only a hybrid nucleic acid molecule having a mutated first sequence made it possible to screen interfering nucleic acid molecules by comparing the effect of an interfering nucleic acid in the presence of a hybrid nucleic acid molecule having a first, unmutated, sequence (SEQ ID NO: 136).

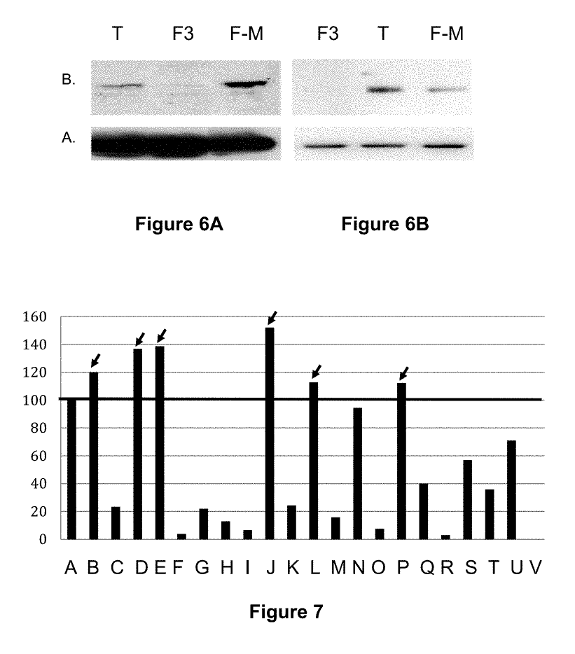

The results obtained by Western blot are represented in FIGS. 6A and 6B.

It is observed from this experiment that the siRNA FLAG-M (SEQ ID NO: 154) makes it possible to detect an increase in the level of expression of CD1 only when the hybrid nucleic acid molecule comprises a mutation in its first sequence (FIG. 6A) but not when the first sequence is unmutated (FIG. 6B).

b) Mutation by G->T Substitution

In a second series of experiments, the inventors carried out screening of interfering molecules according to the invention using the hybrid nucleic acid molecule SEQ ID NO: 121. In this, the first sequence is CCAGCCATGt (SEQ ID NO: 52).

As siRNA transfection control, the negative control siRNAs (“scramble”; T.) are also transfected. These siRNAs have the following sense sequence: 5′-UUCUCCGAACGUGUCACGUtt-3′ (SEQ ID NO: 161) and the complementary strand has the following sequence: 5′-ACGUGACACAUUCGGAGAAtt-3′ (SEQ ID NO: 162).

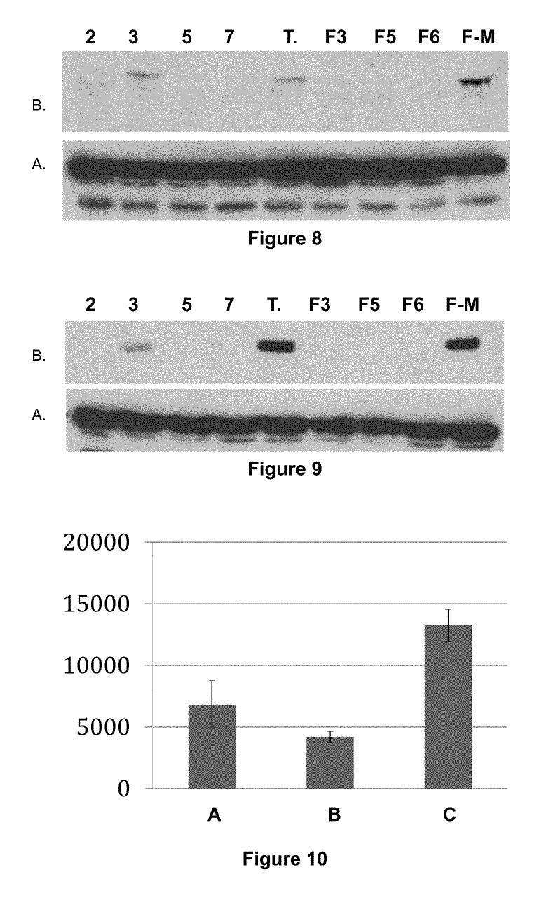

The results obtained by Western blot are represented in FIG. 8.

From this figure it is observed that from the different siRNAs tested, the siRNA FLAG-M (SEQ ID NO: 154) make it possible to detect a higher level of expression of the marker peptide CD1 than the level observed with an irrelevant control. The same results are therefore observed as those obtained for the hybrid nucleic acid molecule SEQ ID NO: 120.

The inventors confirmed that only a hybrid nucleic acid molecule having a mutated first sequence made it possible to screen interfering nucleic acid molecules by comparing the effect of an interfering nucleic acid in the presence of a hybrid nucleic acid molecule having a first, unmutated, sequence (SEQ ID NO: 136).

The results obtained by Western blot are represented in FIG. 9.

It is observed from this experiment that the siRNA FLAG-M (SEQ ID NO: 154) makes it possible to detect an increase in the level of expression of CD1 only when the hybrid nucleic acid molecule has a mutation in its first sequence.

In conclusion, only hybrid nucleic acid molecules comprising a mutated first sequence, especially mutated by a G->T substitution or an AT dinucleotide insertion, makes it possible to screen interfering nucleic acid molecules which increase expression.

### Example 5

### Example of Screening Using FRET as Detection Means

The cells stably expressing the construct SEQ ID NO: 120 were seeded into 24-well plates in the morning, in order to achieve 60% confluence by the evening of the transfection with the siRNAs. In the evening, the cells were transfected with lipofectamine (RNAimax—manufacturer's procedure) at a rate of 10 nM of siRNA per well. Different siRNAs (see example 4) were tested in order to evaluate their respective impacts on the expression of the transgene of interest. The following morning, the cells were washed with 1× PBS, lyzed in 100 microliters of buffer (10 mM TRIS pH=8, 1 mM EDTA, 0.05% NP-40, +protease inhibitors), collected in Eppendorf tubes then subjected to sonication at a rate of 5 cycles composed of 15 seconds of active sonication (maximum power) followed by 15 seconds pause, in a Diagenode sonication bath. The cell lysates are then centrifuged for 5 minutes at 15 000 rcf at 4° C.

The supernatant is recovered and after adjustment to similar concentrations of DNA (and/or protein) of each of the samples (after measuring the DNA concentration by nanodrop quantification, or the protein concentration by the Bradford method) at an amount of 100 micrograms of DNA per liter, 5 microliters per well are deposited in triplicates in a 384-well dish (Greiner-#784076). A mixture of 5 microliters of donor (CISBIO-#610HATAB) and acceptor (CISBIO-#61 FG2XLB) antibody, according to manufacturer (CISBIO)'s instructions, is added to each of the wells followed by incubation away from light at room temperature for 1 hour. The fluorescence arising from the FRET between the donor and the acceptor directed against the TAGs produced by the transgene of interest (FLAG and HA) is read by means of an HTRF apparatus (PHERAstar FS-BMG LABTECH), according to the manufacturer's instructions. After standardization of the data relative to the control which does not have FRET (lysate without TAGs capable of producing a FRET signal), a 10% signal increase compared to the control siRNA (T.) is considered to be significant in terms of increasing the expression of the transgene of interest.

The results are indicated in FIG. 7.

The quantitative FRET results show that the interfering nucleic acid molecules F-N (SEQ ID NO: 146; B), FLAG (SEQ ID NO: 148 and 149; D and E), FLAG Cter (SEQ ID NO: 156; J), F-M (SEQ ID NO: 154; L) and HA3 (SEQ ID NO: 140; P) increase expression.

All of these data show that the method according to the invention makes it possible to screen interfering nucleic acid molecules which increase gene expression.

### Example 6

### Determining the Underlying Mechanism

As mentioned above, it is observed that screening interfering nucleic acid molecules increasing gene expression requires the use of a Kozak sequence having a determined mutation. Such a Kozak sequence has the effect of reducing the expression of the gene it controls, that is to say reducing translation of the protein.

The inventors thus firstly compared the levels of protein expression of the protein cyclin D1, the protein expression of which is controlled by:
the murine cyclin D1 Kozak sequence (mKoz), especially represented by the sequence SEQ ID NO: 12
the murine cyclin D1 Kozak sequence having an AT insertion according to the invention (mKozAT), represented by the sequence SEQ ID NO: 9, and
the optimized cyclin D1 Kozak sequence (KozOPT), of sequence SEQ ID NO: 62.

Murine fibroblast cell lines devoid of the endogenous cyclin D1 gene (Ccnd1^{-/-}) and stably expressing the construct mKoz-Ntag-CycD1 or mKozAT-Ntag-CycD1 (SEQ ID NO: 120) or KozOPT-Ntag-CycD1, were seeded on the morning of the first day and cultured in an incubator at 37° C. with a stable level of CO₂ at 5%, in order to achieve approximately 80% cell confluence by the following day. At this stage, the lines were collected, in order to extract therefrom either the proteins (lysis buffer=10 mM Tris, 1 mM EDTA and 0.05% NP-40), or the total RNAs with Trizol.

The protein lysates were then standardized to an equivalent total protein concentration by the Bradford method, then analyzed by Western blot for actin or cyclin D1. These samples were also analyzed by the Tandem-HTRF method described in example 5.

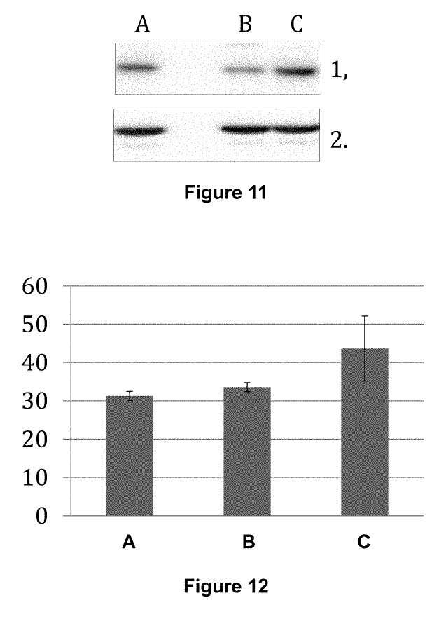

The results obtained are presented in FIG. 10 and FIG. 11.

The results illustrate a reduction in expression resulting from mKozAT compared to the wild-type mKoz sequence or compared to an artificial KozOPT sequence. This therefore means that the mutated Kozak sequence has the effect of reducing protein expression.

The messenger RNAs were used to generate complementary DNA (cDNA) by reverse transcription, then these cDNAs were analyzed by quantitative PCR (qPCR). The content of messenger RNAs resulting from the mKoz-Ntag-CycD1 or mKozAT-Ntag-CycD1 or KozOPT-Ntag-CycD1 constructs was evaluated according to the qPCR following standardization using housekeeping genes (HPRT, B2M, Trfr1, TUBB and GAPDH).

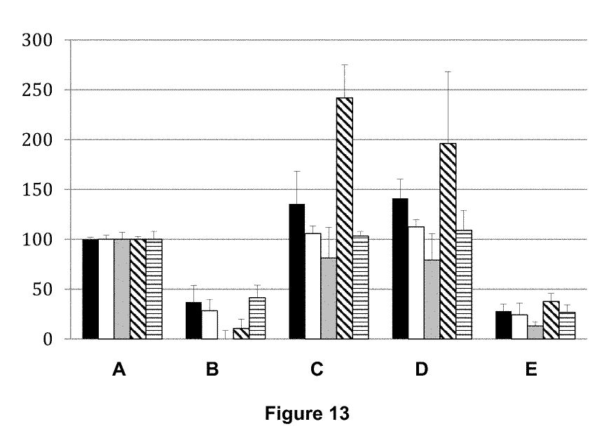

The results are presented in FIG. 12.

A comparable content of messenger RNA appears for Ntag-CycD1 (non-significant difference between the groups, by a Student's test) between the mKoz-Ntag-CycD1 or mKozAT-Ntag-CycD1 or KozOPT-Ntag-CycD1 lines.

Thus, the level of expression is therefore modulated at the translational level.

### Example 7

### Comparison of the Kozak Sequences within the Context of Screening of the siRNAs of the Invention

In order to confirm the importance of the mutated Kozak sequences, the inventors finally tested the effect of different siRNAs (increase or reduction in expression) from different constructs:
N-terminal-tagged cyclin D1 under the control of the murine cyclin D1 Kozak having an AT insertion (Ntag-mKozAT),
N-terminal-tagged cyclin D1 under the control of the murine cyclin D1 Kozak (Ntag-mKoz),
C-terminal-tagged cyclin D1 under the control of the murine cyclin D1 AT Kozak (Ctag-mKozAT),
C-terminal-tagged cyclin D1 under the control of the murine cyclin D1 Kozak having an AT insertion (Ctag-mKozAT), and
N-terminal-tagged cyclin D1 under the control of the murine cyclin D1 Kozak optimized to increase expression (Ntag-KozOPT).

Several siRNAs tested in FIG. 7 were used: SEQ ID NO: 149/150; SEQ ID NO: 155, SEQ ID NO: 154, and SEQ ID NO: 142.

The comparative tests are presented in FIG. 13.

These data show that regardless of the Kozak sequence, an siRNA reducing expression will always be identified as such. On the other hand, siRNAs increasing expression are systematically identified when the reporter is placed under the control of a mutated Kozak sequence (here, having an AT insertion).

All these results confirm the importance of the region regulating translation of the reporter in the screening of the siRNAs which increase gene expression according to the invention.

The invention is not limited to the embodiments presented and other embodiments will become clearly apparent to those skilled in the art.

1-12. (canceled)

13. A method for screening interfering nucleic acids increasing:
gene expression and/or
the activity of genes and/or of ribonucleic acids transcribed from said genes, said interfering nucleic acids having at least partial sequence complementarity with said gene or said RNA, and
said method comprising a step of introducing, into an eukaryotic cell, a hybrid nucleic acid molecule comprising:
a first non-coding sequence intended to initiate translation,
a second sequence at least partially complementary to the sequence of said interfering nucleic acids to be screened,
a third nucleotide sequence encoding at least one determined peptide, said third sequence being under cis translational control of the first sequence,
said first sequence being modified, by substitution, deletion or addition of at least one nucleotide, such that the level of translation of said at least one peptide is reduced by at least 10% relative to the level of translation of said at least one peptide under control of said first sequence in its unmodified version.

14. The method according to claim 13, wherein the eukaryotic cell is capable of RNA interference.

15. The method according to claim 13, wherein the hybrid nucleic acid molecule comprises said first sequence positioned upstream of said third sequence.

16. The method according to claim 13, wherein said nucleic acid molecule is a molecule of deoxyribonucleic acids or a molecule of ribonucleic acids.

17. The method according to claim 16, wherein the nucleic acid molecule is contained in a vector.

18. The method according to claim 13, wherein said first sequence is a Kozak sequence represented, in its unmodified version, by the following sequence:
5′-ssmRccA(T/U)GG-3′ (SEQ ID NO: 1)
wherein R represents a purine, s represents G or C and m represents A/U or C.

19. The method according to claim 13, wherein said first sequence is a Kozak sequence comprising or consisting of, in its modified version, one of the following sequences: SEQ ID NO: 4 or SEQ ID NO: 5

20. The method according to claim 13, wherein said second sequence comprises from 18 to 10 000 nucleotides at least partially complementary to the sequence of said interfering nucleic acids.

21. A hybrid nucleic acid molecule comprising:
a first non-coding sequence intended to initiate translation,
a second sequence at least partially complementary to at least one interfering nucleic acid, and
a third nucleotide sequence encoding at least one determined peptide, said third sequence being under cis translational control of the first sequence,
said first sequence being modified, by substitution, deletion or addition of at least one nucleotide, such that the level of translation of said at least one peptide is reduced by at least 10% relative to the level of translation of said at least one peptide under control of said first sequence in its unmodified version.

22. The hybrid nucleic acid molecule according to claim 21, wherein said nucleic acid molecule is chosen from molecules with the following sequence: SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, and SEQ ID NO: 128.

23. A eukaryotic cell comprising the at least one hybrid nucleic acid molecule as defined in claim 21.

24. A kit, comprising:
the at least one nucleic acid molecule as claimed in claim 21, and
at least one eukaryotic cell.

25. The kit of claim 24, further comprising:
means for transforming a eukaryotic cell by said hybrid nucleic acid molecule.

26. A kit, comprising:
the at least one nucleic acid molecule as claimed in claim 21, and
means for transforming a eukaryotic cell by said hybrid nucleic acid molecule.

27. An intermediate hybrid nucleic acid molecule comprising:
a first non-coding sequence intended to initiate translation,
a third nucleotide sequence encoding at least one determined peptide, said third sequence being under cis translational control of the first sequence
and at least one site for cleavage by a restriction enzyme, enabling the insertion nucleic acid molecule having a sequence complementary to an interfering nucleic acid, said first sequence being modified, by substitution, deletion or addition of at least one nucleotide, such that the level of translation of said at least one peptide is reduced by at least 10% relative to the level of translation of said at least one peptide under control of said first sequence in its unmodified version.
